(19) United States
(12) Patent Application Publication
(10) Pub. No.: US 2012/0052535 A1
(43) Pub. Date: Mar. 1, 2012

(54) PRODUCTION OF TERPENES AND TERPENOIDS IN GLANDULAR TRICHOME-BEARING PLANTS

(76) Inventors: Bernd Markus Lange, Pullman, WA (US); Rigoberto Rios-Estepa, Pullman, WA (US); Glenn W. Turner, Pullman, WA (US)

(21) Appl. No.: 13/256,487

(22) PCT Filed: Mar. 26, 2010
(86) PCT No.: PCT/US2010/028789

§ 371 Date: Nov. 4, 2011

Related U.S. Application Data
(60) Provisional application No. 61/163,829, filed on Mar. 26, 2009.

(51) Int. Cl. C12P 17/06; C12P 5/00; C12P 7/26; C12P 9/00; C12P 17/08; C12P 5/02; C12P 17/04; A01H 5/00; C12P 7/04

(52) U.S. Cl. 435/125; 800/298; 435/166; 435/148; 435/157; 435/124; 435/167; 435/126; 435/131

(57) ABSTRACT

Methods for producing heterologous terpenes, terpenoids and/or small molecules in transgenic glandular trichome-bearing plants are provided, as well as the transgenic glandular trichome-bearing plants capable of producing the heterologous terpenes, terpenoids and small molecules. The genetically engineered glandular trichome-bearing plants contain and express one or more genes which encode proteins active in the biosynthetic pathways which produce the terpenes, terpenoids and small molecules. As a result, the essential oil of the transgenic plant is enriched for the heterologous or homologous terpenes, terpenoids and/or small molecules Storage of the essential oil in the glandular trichomes of the plant reduces the volatility and cytotoxic capacity of the heterologous molecules, thereby increasing yield and decreasing damage to the transgenic plant.

### FIELD

The present disclosure generally relates to the production of homologous or heterologous terpenes and terpenoids, and/or high-value small molecules in genetically engineered glandular trichome-bearing plants. More particularly, the genetically engineered glandular trichome-bearing plants contain and express genes encoding proteins which are active in the biosynthesis of homologous or heterologous terpenes and terpenoids, and/or high-value small molecules.

### BACKGROUND

Most plants have specialized hair-like structures on their leaf surface called trichomes. These structures are involved in a number of adaptive functions including protection from herbivores and microorganisms. There are two main types of trichomes: glandular and non-glandular. Glandular trichomes, which are not as common as non-glandular trichomes, are capable of synthesizing and storing large amounts of secondary metabolites as part of the essential oil of the plant. Essential oil is a volatile, complex mixture characterized by a strong odor that is mainly composed of terpenes. Terpenes and chemically modified forms thereof (generally known as “terpenoids”) are valuable hydrocarbons that are biosynthetically derived from the same basic five-carbon isoprene building blocks.

The biosynthetic pathways that occur in trichomes have several characteristics that make them attractive targets for metabolic engineering. First, trichomes are designed solely for the production of large quantities of specialized small molecules, making them an ideal production system for terpenes and other hydrocarbons derived from terpenes. Second, trichomes are non-essential structures suggesting that modifications to their endogenous pathways would not negatively affect plant health. Third, chemicals that would be toxic to other plant tissues can be produced and enriched in the essential oil of the plant without being cytotoxic to the plant cells due to the trichome's naturally protective structure.

Peppermint is one example of a glandular trichome-bearing plant that can be used as a versatile platform for the production of oils and high-value small molecules. The essential oil distilled from peppermint (Mentha×piperita) leaves is used in numerous consumer products (e.g., chewing gum, toothpaste, and mouthwash), as a flavor in the confectionary and pharmaceutical industries, and as a source of active ingredients for aromatherapy. Peppermint oil consists primarily of p-menthane-type monoterpenes, with smaller amounts of other monoterpenes, and minor quantities of sesquiterpenes (Rohloff, 1999). The essential oil is synthesized and accumulated in specialized peltate glandular trichomes (Gershenzon et al., 1989; McCaskill et al., 1992). These trichomes contain secretory cells, arranged in an eight-celled disk, which are responsible for the synthesis of essential oil. Essential oil is excreted into an emerging cavity formed by the separation of a preformed layer of cuticular material (Amelunxen, 1965). The volatilization of essential oil from peppermint peltate glandular trichomes is negligible (Gershenzon et al., 2000).

Despite the enormous structural diversity represented by isoprenoid natural products, the biochemical principles underlying the biosynthesis of key intermediates are relatively simple. The term isoprenoids is used for compounds formally derived from isoprene (2-methylbuta-1,3-diene), the skeleton of which can usually be discerned in repeated occurrence in any isoprenoid molecule (Ruzicka, 1953). All isoprenoid structures are biosynthetically derived from “active isoprene” (Lynen et al., 1958; Chaykin et al., 1958), isopentenyl diphosphate (IPP), and its isomer dimethylallyl diphosphate (DMAPP) (FIG. 1). Condensation reactions between DMAPP, the starter molecule, and IPP, the chain elongation molecule, yield various prenyl diphosphates, which serve as precursors for terpene synthases and secondary modification enzymes to yield the isoprenoid end products. The universal C5 intermediates IPP and DMAPP can be formed via two different pathways. In yeasts, fungi, archaebacteria and animals the mevalonate (MVA) pathway is responsible for the synthesis of isoprenoid intermediates, whereas an MVA-independent pathway operates in most eubacteria. Both pathways occur in plants and certain algae, where the MVA pathway enzymes are present in the cytosolic/ER compartment and the enzymes of the MVA-independent pathway are localized to plastids (Lange et al., 2000a). Since high levels of both IPP and DMAPP are produced in peppermint glandular trichomes (McCaskill and Croteau, 1995), there is potential for utilizing these trichomes as “green factories” for producing various terpenes and terpenoids derived from the IPP and DMAPP precursors.

While the concept of utilizing plants as “green factories” for the production of small molecules has been of great interest, several issues have plagued metabolic engineering efforts thus far: (1) when terpenes and terpenoids are accumulated in a non-specific fashion, their accumulation causes cytotoxicity; (2) when terpenoids are produced in a non-specific manner, they are generally converted to conjugates for storage, resulting in low accumulation levels; and (3) when produced in most plants, terpenoids are emitted as volatiles, which results in low accumulation levels.

### SUMMARY

An embodiment of the invention is based on the first successful production of novel heterologous terpenes and/or terpenoids in genetically engineered glandular trichome-bearing plants. Trichome-bearing plant species have naturally evolved the capacity to store large amounts of essential oil. The results described herein show that heterologous terpenes and terpenoids (terpenes and terpenoids not naturally produced by the plant) are produced and accumulate in the essential oil of the transgenic plants. The transgenic plants are produced by transformation with one or more genes active in the biosynthesis of the heterologous terpenes and/or terpenoids. Typically, the biosynthetic pathway is normally or “in nature” present in plants of another species, but is not normally (in nature) found or is not operative in the glandular trichome-bearing plants that are genetically engineered according to the invention. By way of example, several heterologous monoterpenes and sesquiterpenes have been produced and accumulated in transgenic mint plants by transforming the plants with one of the following genes from Artemisia annua.: the amorpha-1,4-diene synthase (ADS) gene, (−)-linalool synthase, (+)-limonene synthase, (−)-limonene 7-hydroxylase, or gamma-humulene synthase. The heterologous monoterpenes and sesquiterpenes that were produced in the transgenic plants included amorpha-1,4-diene, (−)-linalool, (+)-limonene, (−)-perillyl alcohol, and gamma-humulene, none of which are normally produced in mint plants. These results show that genetically engineered glandular trichome-bearing plants are suitable hosts for the production of valuable heterologous terpenes and terpenoids. Glandular trichome-bearing plants may also be utilized for the production of other valuable small molecules, for example, small molecules that are derived from the terpenoid or phenylpropanoid biosynthetic pathways, such as abietadiene, amorpha-1,4-diene, 5-epi-aristolochene, artemisinic acid, dehydroartemisinic acid, artemisinin, trans-alpha-bergamotene, beta-bisabolene, alpha- and gamma-bisabolene, (+)-bornyl diphosphate, delta-cadinene (−)-camphene, (+)-3-carene, alpha- and beta-caryophyllene, casbene, ent-cassa-12,15-diene, epi-cedrol, chrysanthemyl diphosphate, 1,8-cineole, (−)-copalyl diphosphate, ent-copalyl diphosphate, beta-cubebene, cubebol, elisabethatriene, beta-eudesmol farnesol, alpha- and beta-farnesene, geraniol, geranyllinalool, germacradienol/geosmin, germacrene A, C, and D, gossypol, alpha-gurjunene, (+)-5(6),13-halimadiene-15-ol, alpha-, beta- and gamma-humulene, epi-isozizaene, ent-kaurene, levopimaradiene (−)-limonene, (−)-isopiperitenol, (+)-limonene, (−)-linalool, longifolene, p-menthane-3,8-diol, (+)-menthofuran, (−)-menthone, (−)-menthone, cis-muuroladiene, myrcene, E-nerolidol, nootkatone, beta-ocimene, patchoulol, pentalenene, beta-phellandrene, (−)-perillyl alcohol, pimara-9(11),15-diene, syn-pimara-7,15-diene, alpha- and beta-pinene, (+)-pulegone, cis-rose oxide, ent-sandaracopimaradiene, delta-selinene, stemar-13-ene, stemodene, terpenticin, gamma-terpinene, alpha-terpineol, terpinolene, tetrahydrocannabinoic acid, trichodiene, (+)-valencene, verbenone, vetispiradiene, alpha-vetivone, viridiflorol, and alpha-zingiberene. In some embodiments of the invention, the terpenes and/or terpenoids are derivatives of precursors of the terpene biosynthetic pathway, examples of which include but are not limited to isopentenyl diphosphate, dimethylallyl diphosphate, geranyl diphosphate, farnesyl diphosphate, geranylgeranyl diphosphate, and squalene. In addition, manipulation of the genetic components of glandular trichome-bearing plants by genetic engineering, for example, to contain and express genes encoding one or more enzymes that catalyze various modification reactions of interest, can result in the production of particular compounds of interest with desired chemical compositions and properties.

In some embodiments, the invention provides a genetically engineered glandular trichome-bearing plant (e.g. a mint plant) comprising one or more expressible genes which encode one or more proteins active in biosynthesis of at least one or more heterologous or homologous terpenes or terpenoids, wherein said heterologous or homologous terpenes or terpenoids are synthesized in glandular trichomes of said genetically engineered glandular trichome-bearing plant and stored in oil of said glandular trichomes of said genetically engineered glandular trichome-bearing plant. Examples of expressible genes include amorpha-1,4-diene synthase, (−)-linalool synthase, (+)-limonene synthase, (−)-limonene 7-hydroxylase or gamma-humulenesynthase. Examples of heterologous or homologous terpenes include monoterpenes, sesquiterpenes, diterpenes, triterpenes, and polyterpenes, such as, e.g. amorpha-1,4-diene, (−)-linalool, (+)-limonene, (−)-perillyl alcohol and/or gamma-humulene.

In other embodiments, the invention provides a method of producing one or more terpenes and terpenoids by i) selecting a glandular trichome-bearing plant (e.g. a mint plant); and ii) genetically engineering the glandular trichome-bearing plant to contain and express one or more genes which encode one or more proteins active in biosynthesis of at least one or more terpenes and terpenoids. The terpenes and terpenoids are synthesized in glandular trichomes of the glandular trichome-bearing plant and stored in oil of the glandular trichomes of the glandular trichome-bearing plant. Examples of suitable genes include amorpha-1,4-diene synthase, (−)-linalool synthase, (+)-limonene synthase, (−)-limonene 7-hydroxylase or gamma-humulenesynthase. Examples of heterologous or homologous terpenes include monoterpenes, sesquiterpenes, diterpenes, triterpenes, and polyterpenes, such as, e.g. amorpha-1,4-diene, (−)-linalool, (+)-limonene, (−)-perillyl alcohol and/or gamma-humulene. In some embodiments, the glandular trichome-bearing plant further comprises RNA sequences that inhibit expression of one or more enzymes of one or more biosynthetic pathways in the glandular trichome-bearing plant. In other embodiments, the invention also provides a method of producing one or more terpenes and terpenoids by growing the genetically engineered glandular trichome-bearing plants of the invention and recovering one or more terpenes and terpenoids from the oil of said glandular trichomes of the glandular trichome-bearing plants. This method may include the step of treating the genetically engineered glandular trichome-bearing plants with one or more jasmonates.

### DESCRIPTION OF THE DRAWINGS

FIGS. 1A and B. A, Outline of the plastidial mevalonate-independent pathway that supplies precursors for monoterpene biosynthesis in peppermint. The following enzymes are involved in this pathway: (1) 1-deoxy-D-xylulose 5-phosphate synthase; (2) 1-deoxy-D-xylulose 5-phosphate reductoisomerase; (3) 2C-methyl-D-erythritol 4-phosphate cytidyltransferase; (4) 4-(cytidine 5′-diphospho)-2C-methyl-D-erythritol 4-phosphate kinase; (5) 2C-methyl-D-erythritol 2,4-cyclodiphosphate synthase; (6) (E)-4-hydroxy-3-methyl-but-2-enyl diphosphate synthase; (7) (E)-4-hydroxy-3-methyl-but-2-enyl diphosphate reductase; (8) isopentenyl diphosphate isomerase; (9) geranyl diphosphate synthase. “Lpl” stands for leucoplast, the intracellular location of the reaction. B, Outline of p-menthane monoterpene metabolism in peppermint glandular trichomes. The following enzymes are involved in this pathway: (1) (−)-limonene synthase; (2) (−)-limonene 3-hydroxylase; (3) (−)-trans-isopiperitenol dehydrogenase; (4) (−)-trans-isopiperitenone reductase; (5) (+)-cis-isopulegone isomerase; (6) (+)-menthofuran synthase; (7a) (+)-pulegone reductase ((−)-menthone-forming activity); (7b) (+)-pulegone reductase ((+)-isomenthone-forming activity); (8a) (−)-menthone: (−)-menthol reductase ((−)-menthol-forming activity); (8b) (−)-menthone: (−)-menthol reductase ((+)-neoisomenthol-forming activity); (9a) (−)-menthone: (+)-neomenthol reductase ((+)-neomenthol-forming activity); (9b) (−)-menthone: (+)-neomenthol reductase ((+)-isomenthol-forming activity). “Lpl”=leucoplast; “ER”=endoplasmic reticulum; “Mit”=mitochondria; “Cyt”=cytosol (intracellular locations of the reactions). The inhibition of (+)-pulegone reductase by (+)-menthofuran is indicated by an arc.

FIGS. 2A-D. Expression patterns of genes involved in peppermint monoterpene biosynthesis, as determined by real-time quantitative PCR, using the peppermint β-actin gene (AW255057) as an endogenous control. The average signal intensity of RNA obtained with 30 d samples (wild-type plants grown under greenhouse conditions) was used as a calibrator (based on prior knowledge expression levels of genes involved in monoterpene biosynthesis are consistently low (but detectable) at this stage of leaf development). The following abbreviations and acronyms are used: DXS, deoxy-D-xylulose 5-phosphate synthase; DXR, 1-deoxy-D-xylulose 5-phosphate reductoisomerase; CMK, 4,4-(cytidine 5′-diphospho)-2C-methyl-D-erythritol 4-phosphate kinase; HDS, (E)-4-hydroxy-3-methyl-but-2-enyl diphosphate synthase; LS, (−)-limonene synthase; L3H, (−)-limonene 3-hydroxylase; PR, (+)-pulegone reductase; MFS, (+)-menthofuran synthase. A, greenhouse controls; B, low light intensity; C, low water treatment; D, low light intensity and high night temperature.

FIG. 3A-F. Experimentally determined monoterpene profiles of greenhouse-grown wild-type (A) and MFS7 transgenic plants (B), and for wild-type plants grown under (C) low water, (D) low light, and (E) a combination of low light and high night temperature conditions. X axis is days after leaf emergence; Y axis is monoterpenes (μg per leaf). The following symbols are used for indicating monoterpene profiles: (−)-limonene, diamond; (+)-pulegone, checkmark within square; (+)-menthofuran, plus sign within square; (−)-menthone, square; (−)-menthol, triangle. Panel F summarizes data on glandular trichome density and size distribution (n=5), as well total essential oil yield at 30 d after leaf emergence (n=3).

FIG. 4. Effect of methyl jasmonate (MeJA) treatment on essential oil yield in peppermint leaves. Abbreviations and acronyms: Con=untreated control plants; MeJA=plants treated with MeJA.

FIG. 5A-C. Gas chromatography (GC) chromatogram of essential oil obtained from A, a non-transgenic control plant, B, a transgenic line expressing amorphadiene synthase, and C, an authentic standard mix containing the antimalarial drug precursor, amorphadiene (lower panel). Amorphadiene accumulated at roughly 8% of the essential oil in transgenic plants, whereas non-transgenic control plants did not contain any detectable levels of this metabolite. The identity of the novel metabolite in transgenic plants was confirmed by gas chromatography/mass spectroscopy (GC-MS) analyses (comparison with mass spectrum of authentic standard).

### DETAILED DESCRIPTION

For the first time, a transgenic glandular trichome-bearing plant has been successfully genetically engineered to produce heterologous terpenes and terpenoids. In particular, exemplary transgenic mint plants that produce and accumulate amorpha-1,4-diene, (−)-linalool, (+)-limonene, (−)-perillyl alcohol and gamma-humulene, have been made, as a result of plant transformation with a gene encoding a protein active in the biosynthetic pathway that produces the terpenes, namely amorpha-1,4-diene synthase (ADS), (−)-linalool synthase, (+)-limonene synthase, (−)-limonene 7-hydroxylase or gamma-humulene synthase, respectively. The transgenic plants accumulated the heterologous terpenes and terpenoids without suffering deleterious effects or loss of yield by volatilization, likely because the terpenes were sequestered in glandular trichomes of the plants. These results demonstrate the feasibility of using genetically engineered glandular trichome-bearing plants as hosts for the production of terpenes and terpenoids. The invention encompasses methods of producing homologous and heterologous terpenes and terpenoids (and related derivatives) in genetically engineered trichome-bearing plants that contain and express genes encoding one or more proteins active in the biosynthesis of the homologous or heterologous terpenes and terpenoids, as well as the genetically engineered glandular trichome-bearing plants themselves, and progeny thereof.

Generally, the methods of the invention are practiced in a plant with glandular trichomes, examples of which include but are not limited to plants from the genus Capsicum, Carum, Gossypium, Humulus, Jasminum, Lavandula, Matricaria, Mentha, Nepeta, Ocimum, Origanum, Perilla, Pogostemon, Rosmarinus, Salvia, Solanum, Thymus, etc.

In some embodiments, the glandular trichome-bearing plant is a mint plant, for example, a mint plant of the genus Mentha. Species of mint that may be utilized in the practice of the invention include but are not limited to Mentha aquatica, Mentha arvensis, Mentha asiatica, Mentha australis, Mentha canadensis, Mentha cervina, Mentha citrata, Mentha crispata, Mentha cunninghamia, Mentha dahurica, Mentha diemenica, Mentha gattefossei, Mentha grandiflora, Mentha haplocalyx, Mentha japonica, Mentha kopetdaghensis, Mentha laxiflora, Mentha longifolia, Mentha sylvestris, Mentha piperita, Mentha pulegium, Mentha requienii, Mentha sachalinensis, Mentha satureioides, Mentha spicata, Mentha suaveolens, or Mentha vagans. Mint cultivars may also be used, examples of which include but are not limited to Water mint, Marsh mint, Ginger mint, Corn Mint, Wild Mint, Japanese Peppermint, Field Mint, Pudina, Asian Mint, Australian mint, Hart's Pennyroyal, Bergamot mint, Wrinkled-leaf mint, Dahurian Thyme, Slender mint, Forest mint, Horse Mint, Pennyroyal, Corsican mint, Garden mint, Native Pennyroyal, Spearmint, Curly mint, Apple mint, Pineapple mint, Erospicata, or Gray mint.

The glandular trichome-bearing plant of the invention is genetically engineered. By “genetically engineered” we mean that the genetic material of the plant (e.g. DNA, RNA etc.) has been altered or modified, compared to the genetic material of the plant before it was genetically engineered according to the present invention. The plant that is so genetically engineered may be a native or “wild type” plant, or may be a plant that has previously been (or is concurrently) genetically engineered in some manner (e.g. to exhibit resistance to disease, pesticides, difficult growth conditions such as drought; or to contain inhibitory RNA that blocks production of one or more proteins or enzymes; etc.). Alternatively, the plant that is genetically engineered by the methods of the invention may be a plant that is a cross or hybrid of other plant varieties, species, etc., either a naturally occurring hybrid or one that has been purposefully bred, e.g. by selecting and crossing two varieties or species. Both genetically engineered plants and progeny thereof are encompassed by the invention.

In some embodiments of the invention, the genetic engineering that is carried out modifies the plant by causing it to contain (and usually, to express or overexpress) genetic material that is the same or similar to that which is already present in the plant (i.e. homologous genetic material), but which, after genetic engineering, is present in a different amount or form, e.g. additional copies of a gene of interest may be introduced, or mutations may be introduced into the existing genetic material of the plant, etc. The products made in the plant as a result of the introduction of such homologous sequences (e.g. terpenes and/or terpenoids) are referred to as homologous products, e.g. homologous terpenes and terpenoids.

In other embodiments of the invention, the genetic engineering that is carried out modifies the plant by causing it to contain (and usually, to express or overexpress) genetic material that is not normally found in the plant, resulting in production of a transgenic plant. By “transgenic” we mean a plant (or progeny thereof) that has been genetically engineered to contain and express one or more heterologous nucleic acid sequences of interest, i.e. nucleic acid sequences that are not found in the plant in nature. Examples of such nucleic acids include but are not limited to: sequences that encode or contain genes encoding proteins or peptides (which may be referred to as transgenes, foreign genes, heterologous genes, passenger genes, etc.); silencing or inhibiting RNA; sequences encoding tRNA; sequences encoding various genetic elements such as promoter, enhancer and other transcription and/or translation controlling sequences; etc. Such heterologous nucleic acid sequences originate from another organism, e.g. from another plant species or variety, or even from a non-plant species. In some embodiments, the heterologous nucleic acid sequences encode proteins, frequently enzymes, that are active in (i.e. participate in, and may be required or necessary for) the biosynthesis of terpenes and/or terpenoids of interest, but which are not normally (in nature) found in or produced by the plant. For example, the proteins may be enzymes that catalyze one or more steps in a terpene or terpenoid biosynthetic pathway. These products are referred to as heterologous products, e.g. heterologous terpenes and/or terpenoids. The heterologous proteins or enzymes may participate directly in the biosynthetic pathway of terpene/terpenoid production, or may modulate the biosynthesis in an indirect manner, e.g. by participating in and increasing activity of a competing pathway, by catalyzing the formation of a precursor that then enters a terpene/terpenoid biosynthetic pathway, etc.

Generally, genetic engineering of the plants is carried out in a manner that results in incorporation of DNA comprising one or more nucleic acid sequences of interest (frequently genes) into the chromosomes of the plant, although this need not always be the case. The DNA might also reside in or be part of an extrachromosomal element. Within the genetically engineered plant, the genes are expressible, i.e. they are associated with (operably linked to) other suitable genetic elements such as promoters, enhancers, etc., in a manner that allows or causes or promotes transcription of the gene into RNA (e.g. mRNA) within the plant. Transcription is typically followed by successful translation of an active form of the protein or enzyme, except when the gene encodes an RNA that is intended to function as an inhibitor, such as iRNA or siRNA. In addition, the nucleic acids of interest which are introduced into the genetically engineered plant may contain genetic sequences encoding factors that control the expression of other genes.

The glandular trichome-bearing plant may be transformed using any of the many methods that are known in the art. For example, Agrobacterium, Sinorhizobium, Mesorhizobium, or Rhizobium-mediated transformation methods, as are known in the art, may be used. (For example, Broothaerts et al., 2005; Gelvin et al., 2005 for descriptions of plant transformation techniques). Alternatively, other methods are also known for transforming plants, including but not limited to: particle bombardment using small metal, e.g. gold or tungsten, particles (or other small particles) coated with DNA which are shot into young plant cells or plant embryos; electroporation, whereby transient holes are made in plant cell membranes using electric shock, allowing DNA to enter; and viral transduction, in which the desired genetic material is packaged into a suitable plant virus and the modified virus is allowed to infect the plant. In this latter case, if the genetic material is DNA, it can recombine with the chromosomes to produce transformant cells. However genomes of most plant viruses consist of single stranded RNA which replicates in the cytoplasm of infected cell. For such genomes this method is a form of transfection and not a real transformation, since the inserted genes never reach the nucleus of the cell and do not integrate into the host genome. The progeny of the infected plants is virus free and also free of the inserted gene. Thus, gene expression is confined to the transfected plant and not passed to the next generation.

The trichome-bearing plant(s) is genetically engineered to contain one or more genes coding, for example, for a protein involved in the synthesis of a terpene or terpenoid of interest. The one or more genes may be over-expressed, i.e. expressed at a level that is higher or greater than that which is typically observed or attained when the gene is present in its natural or native host. Expression of the one or more genes is generally driven by a promoter (and possibly other control elements), and the promoter/control elements may be naturally associated with the gene (e.g. the promoter/control elements drive and/or modulate expression of the gene in the plant or organism from which the gene originates, i.e. the organism where the gene is found in nature). Alternatively, heterologous promoters and control elements may be employed in combination with the gene. Examples of promoters that may be employed in the practice of the invention include but are not limited to various cell type or tissue-specific promoters (examples of which include but are not limited ubiquitous promoters (active in substantially all tissues or cells of an organism; examples of which include but are not limited to cauliflower mosaic viruss 35S promoter, ubituitin promoter, actin promoter, alcohol dehydrogenase promoter; Gelvin, 2005) and cell type or tissue-specific promoters (examples of which include but are not limited to trichome-specific promoters (Wang et al., 2002; Gutierrez-Alcala et al., 2005; Shangguang et al., 2008).

In one embodiment of the invention, the one or more genes are “antisense” to the sequence of one or more target genes of interest in the plant that is genetically engineered. Expression of genes that are “antisense” to the target genes can, for example, be used to knock-down or knock-out the expression of a target gene through RNA interference (RNAi). Expression of such RNA decreases or eliminates expression of one or more target genes. This strategy may be used, for example, to decrease or eliminate unwanted activity of an enzyme that otherwise interferes with a terpene or terpenoid biosynthetic pathway, e.g. interferes by competing for substrates required for terpene/terpenoid synthesis, or by producing a substance that inhibits terpene/terpenoid synthesis, or that causes unwanted modification or catalysis of terpenes/terpenoids, etc. RNAi can reduce or eliminate this activity.

In some embodiments, the glandular trichome-bearing plant is genetically engineered and produces essential oils, which may be stored, for example, in the glandular trichomes of the plant, and the essential oil may contain one or more homologous or heterologous terpenes or modified terpenes (e.g. terpenoids). The one or more homologous or heterologous terpenes/terpenoids may include but are not limited to: hemiterpene (one isoprene unit) and oxygen containing terpenoid derivatives thereof (hemiterpenoids) such as prenol and isovlaeric acid, etc.; monoterpenes (two isoprene units) such as geraniol, limonene and terpineol, etc.; sesquiterpenes (three isoprene units) such as farnesenes and farnesol, etc.; diterpenes (four isoprene units) such as cafestol, kahweol, cembrene, taxadiene, etc.; sesterterpenes (five isoprene units e.g. geranylfarnesol, etc.; triterpenes (six isoprene units) such as squalene, etc.; tetraterpenes (eight isoprene units) such as acyclic lycopene, monocyclic gamma carotene and bicyclic alpha- and beta-carotenes; and polyterpenes (long chains of many isoprene units such as gutta-percha (natural latex). The terpene may be in a terpenoid form of the molecule, and may be linear or cyclic. The terpene/terpenoid can be endogenous or exogenous to the glandular trichome-bearing plant. A terpene stored in the essential oil in a glandular trichome may also have a reduced cytotoxic capacity to the glandular trichome cell tissue as compared to other plant cell tissues. The toxicity of terpenoids on plant cell cultures has been demonstrated in numerous publications (Scragg A. H. et al. 1997). Further, the homologous or heterologous terpenes/terpenoids stored in the essential oil in the glandular trichomes may have reduced volatilization.

The over-expression of the one or more genes may increase essential oil production. The over-expression of the one or more genes may alter the essential oil composition as compared to the composition of the wild type plant essential oil. The over-expression of the one or more genes may enrich the essential oil with a terpene/terpenoid of interest, i.e. the terpene/terpenoid of interest is mixed with or stored with or comprises a portion of the essential oil of the plant. The terpene/terpenoid of interest may be at least about 5%, 10%, 15%, 20%, 25%, 30%, 35%, 40%, 45%, 50%, 55%, 60%, 65%, 70%, 75%, 80%, 85%, or 90% of the total essential oil yield of the transgenic trichome-bearing plant. Each leaf of the transgenic trichome-bearing plant may produce at least about 900 μg of essential oil. Good yields in the greenhouse are generally considered to be from about 1,500 to about 2,200 μg of essential oil per leaf. This may translate to yields in the field of > about 100 pounds per acre, depending on the growing area.

In some embodiments of the invention, the genes are genes (which may be transgenes) which include one or more of amorpha-1,4-diene synthase (ADS), (−)-linalool synthase, (+)-limonene synthase, (−)-limonene 7-hydroxylase and/or gamma-humulene synthase. Over-expression of, for example, an ADS transgene may result in production of one or more of amorpha-1,4-diene, (−)-linalool, (+)-limonene, (−)-perillyl alcohol and/or gamma-humulene. The amorpha-1,4-diene, (−)-linalool, (+)-limonene, (−)-perillyl alcohol and/or gamma-humulene may account for at least about 5%, 10%, 15%, 20%, 25%, 30%, 35%, 40%, 45%, 50%, 55%, 60%, 65%, 70%, 75%, 80%, 85%, or 90% of the total essential oil yield of a transgenic trichome-bearing plant.

Further, the one or more genes may code for proteins involved in terpene biosynthesis. Terpene biosynthesis may be hemiterpene, monoterpene, sesquiterpene, diterpene, sesterterpenes, triterpene, tetraterpene, or polyterpene biosynthesis. Specifically, the one or more genes (which may be transgenes) may code, for example, for (+)-bornyl diphosphate synthase; (−)-camphene synthase; (+)-3-carene synthase; chrysanthemyl diphosphate synthase; 1,8-cineole synthase; geraniol synthase; isoprene synthase; (−)-limonene synthase; (+)-limonene synthase; linalool synthase; myrcene synthase; (E)-beta-ocimene synthase; (−)-beta-phellandrene synthase; alpha--pinene synthase; beta-pinene synthase; (+)-sabinene synthase; gamma-terpinene synthase; alpha-terpineol synthase; terpinolene synthase; amorpha-4,11-diene synthase; 5-epi-aristolochene synthase; (E)-beta-bisabolene synthase; (E)-gamma-bisabolene synthase; (+)-delta-cadinene synthase; beta-caryophyllene synthase; epi-cedrol synthase; beta-cubebene synthase; beta-eudesmol synthase; (E,E)-alpha-farnesene synthase; (E)-beta-farnesene synthase; germacradienol/geosmin synthase; germacredienol synthase; germacrene A synthase; germacrene C synthase; germacrene D synthase; (−)-alpha-gurjunene synthase; gamma-humulene synthase; epi-isozizaene synthase; longifolene synthase; cis-muuroladiene sunthase; E-nerolidol synthase; patchoulol synthase; pentalenene synthase; delta-selinene synthase; delta 1-tetrahydrocannabinoic acid; trichodiene synthase; (+)-valencene synthase; vetispiradiene synthase; alpha-zingiberene synthase; abietadiene synthase; abietadiene/levopimaradiene synthase; casbene synthase; ent-cassa-12,15-diene synthase; (−)-copalyl diphosphate synthase; ent-copalyl diphosphate synthase; elisabethatriene; (+)-5(6),13-halimadiene-15-ol synthase; ent-kaurene synthase; levopimaradiene synthase; pimara-9(11),15-diene synthase; syn-pimara-2,15-diene synthase; ent-sandaracopimaradiene synthase; stemar-13-ene synthase; stemodene synthase; terpenticin synthase; geranyllinalool synthase; acyclic monoterpene primary alcohol:NADP+ oxidoreductase; garaniol 10-hydroxylase; (−)-limonene 7-hydroxylase; amorpha-4,11-diene oxidase; artemisinic aldehyde delta 11(13) reductase; abietadienol/abietadienal oxidase; 5-epi-aristolochene 1,3-dihydroxylase; (+)-delta-cadinene 8-hydroxylase; premnaspirodiene oxygenase; taxoid 2-alpha-hydroxylase; taxane 5-alpha-hydroxylase; taxane 13-alpha-hydroxylase; taxane 10-beta-hydroxylase; taxane 14-beta-hydroxylase; taxoid 7-beta-hydroxylase; taxa-4(20),11(12)-diene-5-alpha-ol O-acetyltransferase; taxoid 2-alpha-O-benzoyltransferase; taxoid 10-beta-O-benzoyltransferase; N-benzoyltransferase; phenylalanine aminomutase; C13-phenylpropanoyl-CoA transferase; and/or geranylgeraniol 18-hydroxylase (see Table 1).

TABLE 1
Enzymes Involved in Terpene Biosynthesis and Modification

| | cDNA Source(s) |
|---|---|
| Monoterpene Synthases | |
| (+)-Bornyl diphosphate synthase | Salvia officinalis |
| (−)-Camphene synthase | Abies grandis, Pseudotsuga |
| | menziessii |
| (+)-3-Carene synthase | Picea abies, Salvia stenophylla |
| Chrysanthemyl diphosphate synthase | Chrysanthemum |
| 1,8-Cineole synthase | Arabidopsis thaliana, Nicotiana |
| | suaveolens, Salvia officinalis |
| Geraniol synthase | Cinnamomum tennipilum, |
| | Ocimum basilicum |
| Isoprene synthase | Populus |
| (−)-Limonene synthase | Abies grandis, Mentha spicata, |
| | Mentha x piperita |
| (+)-Limonene synthase | Citrus paradisii, Citrus unshiu, |
| | Schizonepetatemifolia, mutant |
| | enzyme generated from spearmint |
| | (−)-limonene synthase by site- |
| | directed mutagenesis |
| Linalool synthase | Clarkia breweri, Artemisia annua, |
| | Mentha |
| Myrcene synthase | Abies grandis, Ochtodes |
| | secumdiramea, Pinus, Quercus |
| | ilex |
| (E)-beta-Ocimene synthase | Arabidopsis thaliana, |
| | Lotus japonicus |
| (−)-beta-Phellandrene synthase | Abies grandis |
| alpha- and beta-Pinene synthases | Abies grandis, Artemisia annua, |
| | Citrus, Picea sitchensis |
| (+)-Sabinene synthase | Salvia officinalis |
| gamma-Terpinene synthase | Citrus, Thymus vulgaris |
| alpha-Terpineol synthase | Vitis vinifera |
| Terpinolene synthase | Abies grandis |
| Sesquiterpene Synthases | |
| Amorpha-4,11-diene synthase | Artemisia annua |
| 5-epi-Aristolochene synthase | Nicotiana tabacum, |
| | Penicillium roquefortii |
| (E)-beta-Bisabolene synthase | Abies grandis, Picea abies |
| (E)-gamma-Bisabolene synthase | Pseudotsuga menziessii |
| (+)-delta-Cadinene synthase | Gossypium sp. |
| beta-Caryophyllene synthase | Arabidopsis thaliana, Artemisia |
| | annua, Oryza sativa |
| epi-Cedrol synthase | Artemisia annua |
| beta-Cubebene synthase | Magnolia grandiflora |
| beta-Eudesmol synthase | Zingiber zerumbat |
| (E,E)-alpha-Farnesene synthase | Malus, Picea abies |
| (E)-beta-farnesene synthase | Mentha x piperita, Malus, |
| | Picea abies |
| Germacradienol/geosmin synthase | Streptomyces avermitilis |
| Germacredienol synthase | Streptomyces coelicolor |
| Germacrene A synthase | Artemisia annua, Lactuca sativa, |
| | Solidago canadensis |
| Germacrene C synthase | Lycopersicon esculentum |
| Germacrene D synthase | Vitis vinifera |
| (−)-alpha-Gurjunene synthase | Solidago canadensis |
| gamma-Humulene synthase | Abies grandis |
| epi-Isozizaene synthase | Streptomyces coelicolor |
| Longifolene synthase | Picea abies |
| cis-Muuroladiene sunthase | Mentha x piperita |
| E-Nerolidol synthase | Antirhinum majus, Zea mays |
| Patchoulol synthase | Pogostemon cablin |
| Pentalenene synthase | Streptomyces strain UC5319 |
| delta-Selinene synthase | Abies grandis |
| Delta1-Tetrahydrocannabinoic acid | Cannabis sativa |
| Trichodiene synthase | Fusarium sporotrichoides |
| (+)-Valencene synthase | Vitis vinifera, Citrus |
| Vetispiradiene synthase | Hyoscyamus muticus |
| alpha-Zingiberene synthase | Ocimum basilicum |
| Diterpene Synthases | |
| Abietadiene synthase | Abies grandis |
| Abietadiene/levopimaradiene | Abies grandis |
| synthase | |
| Casbene synthase | Ricinus communis |
| ent-Cassa-12,15-diene synthase | Oryza sativa |
| (−)-Copalyl diphosphate synthase | Arabidopsis thaliana, Pisum |
| | sativum, Stevia rebaudiana, |
| | Zea mays |
| ent-Copalyl diphosphate synthase | Scoparia |
| Elisabethatriene | Pseudopterogorgia elisabethae |
| (+)-5(6),13-Halimadiene-15-ol | Mycobacterium tuberculosis |
| synthase | |
| ent-Kaurene synthase | Cucurbita |
| Levopimaradiene synthase | Ginkgo biloba |
| Pimara-9(11),15-diene synthase | Streptomyces sp. KO-3988 |
| syn-Pimara-7,15-diene synthase | Oryza sativa |
| ent-Sandaracopimaradiene synthase | Oryza sativa |
| Stemar-13-ene synthase | Oryza sativa |
| Stemodene synthase | Oryza sativa |
| Terpenticin synthase | Streptomyces griseolosporeus |
| | sp. MF 730-N6 |
| Homoterpene Synthases | |
| Geranyllinalool synthase | Arabidopsis thaliana |
| Monoterpene Substitutions and | |
| Redox Modifications | |
| Genes/enzyme of the peppermint | |
| monoterpene pathway | |
| Acyclic monoterpene primary | Rauwolfia serpentina |
| alcohol:NADP+ oxidoreductase | |
| CYP170A1 (albaflavenone | Streptomyces coelicolor |
| biosynthesis) | |
| Garaniol 10-hydroxylase | Catharanthus roseus |
| (−)-Limonene 7-hydroxylase | Perilla frutescens |
| Sesquiterpene Substitutions and | |
| Redox Modifications | |
| Artemisinin pathway | |
| Amorpha-4,11-diene oxidase | Artemisia annua |
| (CYP71AV1) | |
| Artemisinic aldehyde Delta11(13) | Artemisia annua |
| reductase | |
| Abietadienol/abietadienal oxidase | Pinus taeda |
| 5-epi-Aristolochene 1,3- | Nicotiana tabacum |
| dihydroxylase | |
| (+)-delta-Cadinene 8-hydroxylase | Gossypium |
| Premnaspirodiene oxygenase | Hyoscyamus muticus |
| Diterpene Substitutions and Redox | |
| Modifications | |
| Taxol pathway | |
| Taxoid 2alpha-hydroxylase | Taxus cuspidata |
| Taxane 5alpha-hydroxylase | Taxus cuspidata |
| Taxane 13alpha-hydroxylase | Taxus cuspidata |
| Taxane 10beta-hydroxylase | Taxus cuspidata |
| Taxane 14beta-hydroxylase | Taxus cuspidata |
| Taxoid 7beta-hydroxylase | Taxus cuspidata |
| Taxa-4(20),11(12)-diene-5alpha-ol | Taxus cuspidata |
| O-acetyltransferase | |
| Taxoid 2alpha-O- | Taxus cuspidata |
| benzoyltransferase | |
| Taxoid 10beta-O- | Taxus cuspidata |
| benzoyltransferase | |
| N-Benzoyltransferase (taxoid) | Taxus cuspidata |
| Phenylalanine aminomutase | Taxus cuspidata |
| C13-phenylpropanoyl-CoA | Taxus cuspidata |
| transferase | |
| Geranylgeraniol 18-hydroxylase | Croton sublyratus |

Alternatively, or in addition, the one or more genes may code for a protein active in the plastidial mevalonate-independent pathway. Specifically, the one or more genes may code for 1-deoxy-D-xylulose 5-phosphate synthase; 1-deoxy-D-xylulose 5-phosphate reductoisomerase; 2C-methyl-D-erythritol 4-phosphate cytidyltransferase; 4-(cytidine 5′-diphospho)-2C-methyl-D-erythritol 4-phosphate kinase; 2C-methyl-D-erythritol 2,4-cyclodiphosphate synthase; (E)-4-hydroxy-3-methyl-but-2-enyl diphosphate synthase; (E)-4-hydroxy-3-methyl-but-2-enyl diphosphate reductase; isopentenyl diphosphate isomerase; or geranyl diphosphate synthase.

The one or more genes may also code for proteins involved in p-menthane monoterpene metabolism. Specifically, the one or more genes may code for: (−)-limonene synthase; (−)-limonene 3-hydroxylase; (−)-trans-isopiperitenol dehydrogenase; (−)-trans-isopiperitenone reductase; (+)-cis-isopulegone isomerase; (+)-menthofuran synthase; (+)-pulegone reductase; (−)-menthol reductase; and (+)-neomenthol reductase.

Further, the one or more genes may code for DXP synthase (DXPS); (−)-limonene 3-hydroxylase (L3H); or menthofuran synthase (MFS). Expression of an antisense transgene of MFS may result in the reduced production of (+)-menthofuran. The amount of (+)-menthofuran produced may be reduced by about 5%, 10%, 15%, 20%, 25%, 30%, 35%, 40%, 45%, 50%, 55%, 60%, 65%, 70%, 75%, 80%, 85%, 90%, or 95%.

The genetically engineered plants of the invention are generally grown under conditions that are suitable for the expression of the genes contained within, and under conditions that allow production and storage of the homologous or heterologous terpenes/terpenoids of interest. Those of skill in the art will recognize that such conditions include the provision of adequate water, nutrients, light, etc. as well as suitable temperatures. Conditions may vary somewhat, depending on plant species, on which terpenes/terpenoids are being produced, the climate, geography and geology where the plants are grown, available resources, and other factors.

In some embodiments of the invention, glandular trichome-bearing plants (both transgenic and non-transgenic) are treated with chemicals to increase the density of glandular trichomes in the plant. For example, the plant hormone methyl jasmonate, jasmonoyl acid (JA) derivatives (Wasternack, 2007), coronatine, and/or various ethylene releasing agents may be used for the treatment. In one embodiment, the chemicals that are applied include one or more jasmonates, which include but are not limited to: jasmonate esters such as methyl jasmonate; amino acid or peptide conjugates of jasmonyl acid (e.g. including conjugates formed with glycine, alanine, valine, leucine and isoleucine); various thiazole derivatives; 9,10-dihydro-JA and their methyl esters, coronalon and other 6-substituted 4-oxo-indanoyl-isoleucine conjugates, cis-jasmone, etc. The chemicals used for the treatment may be natural or synthetic. In preferred embodiments, the glandular trichome-bearing plant would be treated with a dilute solution of MeJA over a growth period. A working solution of MeJA will typically have a concentration in the range of from about 10 μM to about 10 mM, and may be diluted in, for example, water or ethanol or combinations thereof, or some other suitable solvent, for application to the plant. For example, a 1:4,000 (v:v) MeJA to water solution may be used. Solubilizers and wetting agents may also be combined with the working MeJA solution. The glandular trichome-bearing plant may be treated, for example, about once a week with a volume of about 100 ml/m² over a period of, for example, three weeks. The solution may be applied by misting or spraying the plant, or by any other suitable means. The increase in density of trichomes in the plant will typically be, for example, about 5%, 10%, 15%, 20%, or 25%, compared to untreated control plants, usually resulting in an increase in essential oil production in the treated plant of at least about 5%, 10%, 15%, 20%, or 25% (e.g. 24%) or greater.

The essential oil may be extracted (harvested, recovered, etc.) from the genetically engineered trichome-bearing plant using any of several known suitable methods, including but not limited to steam distillation, organic extraction, and microwave techniques. The total essential oil yield of the genetically engineered trichome-bearing plant and the yield per leaf may be determined. The various chemical components of the essential oil may be isolated through traditional organic extraction and purification methods. Further, the glandular trichome-bearing plant and its essential oil may be subjected to qualitative and quantitative analysis. The composition and quality of the essential oil may be determined using, for example, gas chromatography/mass spectroscopy (GC/MS). Leaves can be directly (without prior freezing) steam-distilled and solvent-extracted using, for example, 10 mL of pentane in a condenser-cooled Likens-Nickerson apparatus (Ringer et al., 2003). Terpenes and other components can then be identified by comparison of retention times and mass spectra to those of authentic standards in gas chromatography with mass spectrometry detection. Quantification can be achieved by gas chromatography with flame ionization detection based upon calibration curves with known amounts of authentic standards and normalization to the peak area of camphor as internal standard.

Terpenes/terpenoids produced by the methods of the invention have many varied uses, e.g. in pharmaceuticals, food products, cosmetics, as pesticides, for the treatment of disease conditions, etc. In addition, the oils may be used as biofuels, either after extraction, or in situ within the leaves.

### EXAMPLES

### Example 1

### Genotype-Dependent and Environmental Effects on Essential Oil Yield Correlate Directly with the Density of Glandular Trichomes

### 1.1 Peppermint as a Model for Essential Oil Production

Efforts to modulate essential oil yield and composition have been successful but further improvements can only be achieved if one can build on an in-depth appreciation of the currently ill-understood processes controlling glandular trichome formation and monoterpene biosynthesis. Mahmoud and Croteau (2001) reported that, by over-expressing the gene encoding 1-deoxy-D-xylulose 5-phosphate reductoisomerase (DXR) in peppermint plants, an up to 1.5-fold essential oil yield increase was observed. Antisense suppression of the (+)-menthofuran synthase (MFS) gene led to a dramatic decrease in the amounts of the undesirable side product (+)-menthofuran. A slight increase in overall monoterpene yields was also reported for transgenic plants with increased expression levels of the gene encoding (−)-limonene synthase (Diemer et al., 2001), whereas only minor effects on yield were detected in an independent study (Krasnyansky et al., 1999). Transgenic plants over-expressing the genes coding for (−)-limonene 3-hydroxylase (L3H) did not accumulate increased levels of the recombinant protein and the composition and yield of the essential oils was the same as in wild-type controls. However, co-suppression of the L3H gene resulted in a vastly increased accumulation of the intermediate (−)-limonene, without notable effects on oil yield (Mahmoud et al., 2004).

A reexamination of the above-mentioned transgenic lines was undertaken to better understand the factors controlling essential oil yield and composition in peppermint. During routine analyses of MFS7 plants (Mahmoud and Croteau, 2001) that had been propagated in the greenhouse for 7 years, significantly elevated essential oil quantities were detected compared to wild-type controls. The data indicated that genotype-dependent and environmental effects on essential oil yield correlate directly with the density of glandular trichomes on the leaf surface. Additionally, the plant hormone methyl jasmonate was identified as a chemical modulator of glandular trichome density and essential oil yield. In addition, a new set of transgenic peppermint plant lines was generated which express the heterologous genes amorpha-1,4-diene synthase from Artemisia annua (Mercke et al., 2000), (−)-linalool synthase from Mentha citrata (Crowell et al., 2002), (+)-limonene synthase (a mutant generated by site-directed mutagenesis of (−)-limonene synthase from Mentha spicata; Colby et al., 1993), (−)-limonene 7-hydroxylase from Perilla frutescens (Mau et al., 2010), and gamma-humulene synthase from Abies grandis (Steele to al., 1998). Some of these transgenic lines accumulated either amorpha-1,4-diene, (−)-linalool, (+)-limonene, (−)-perillyl alcohol or gamma-humulene, which are not produced naturally in peppermint. These results, describe in detail below, indicate that peppermint (and potentially other members of the mint family) might be utilizable as a platform for producing high-value small molecules derived from the terpenoid pathway.

Peppermint trichomes also synthesize phenylpropanoids (Voirin and Bayet, 1992) and it is likely that the production of high-value phenylpropanoids in transgenic mints can be achieved as well. Valuable phenylpropanoids include, but are not limited to, eugenol, chavicol, safrole, estragol (present in essential oils), stilbenes, and flavonoids. The key advantage of peppermint relates to the fact that the production of valuable small molecules is restricted to specialized cells within glandular trichomes. Similar approaches should be adaptable to other plants producing phenylpropanoids in specialized anatomical structures such as glandular trichomes, secretory cavities, laticifers, resin blisters, and resin ducts.

1.2 Biosynthetic Gene Expression Patterns Correlate with Monoterpenoid Essential Oil Composition but not with Yield

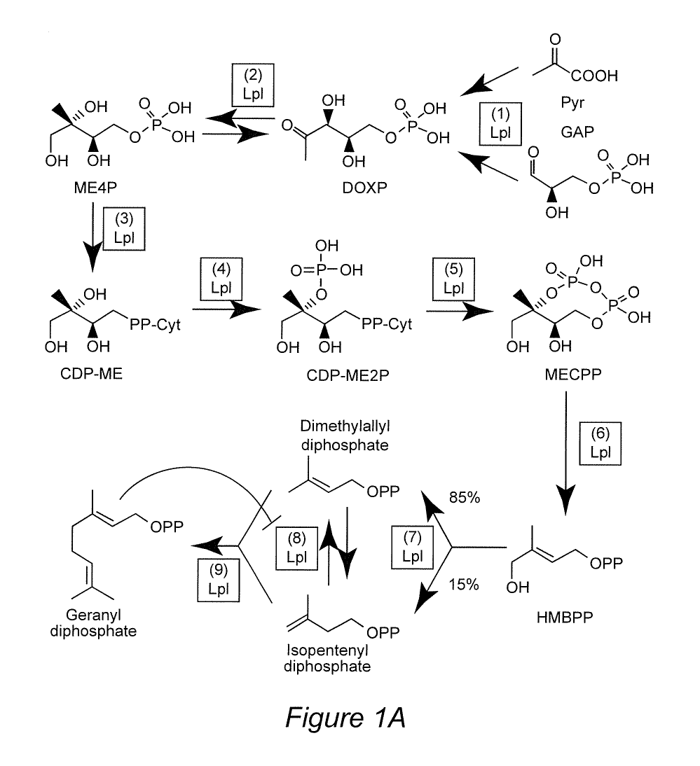

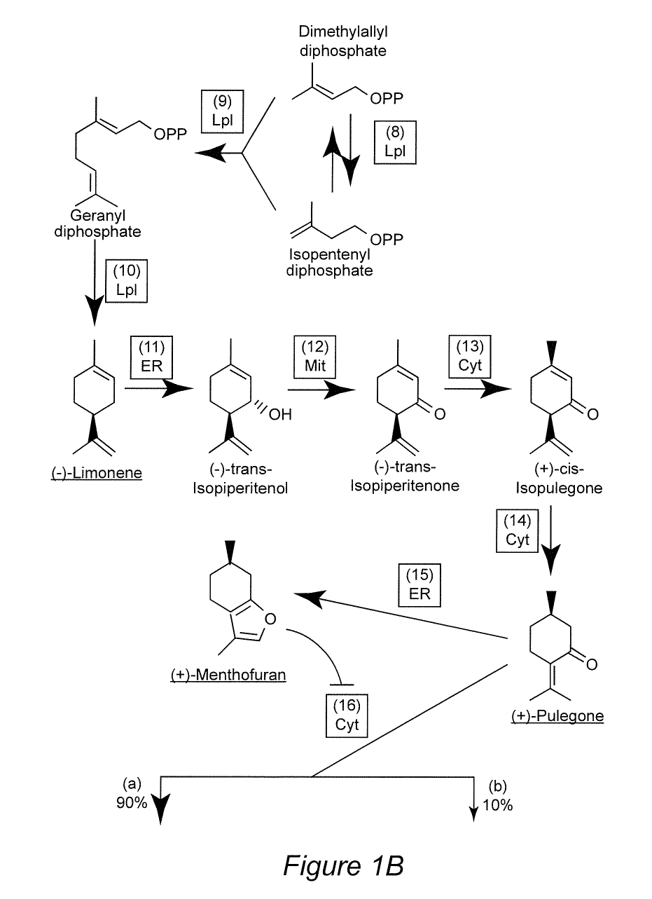

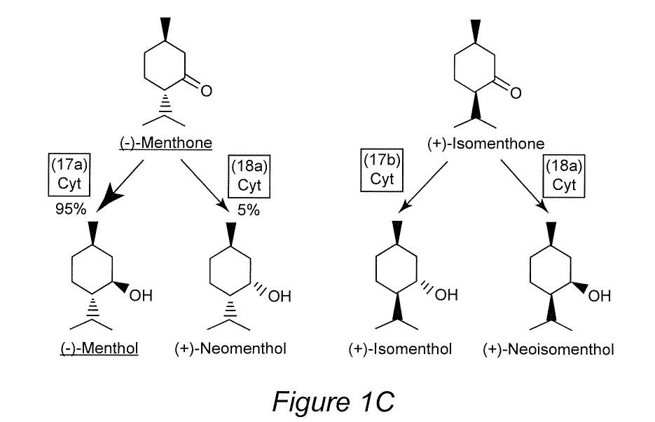

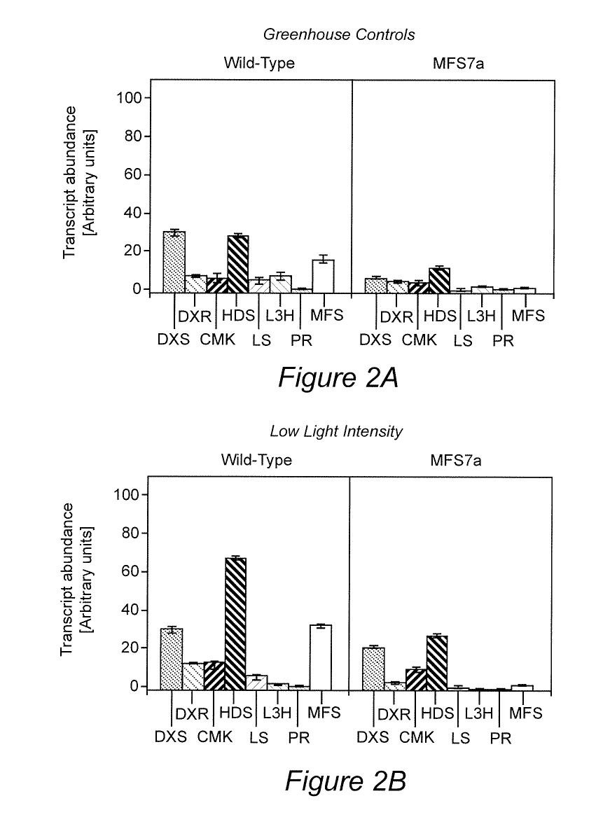

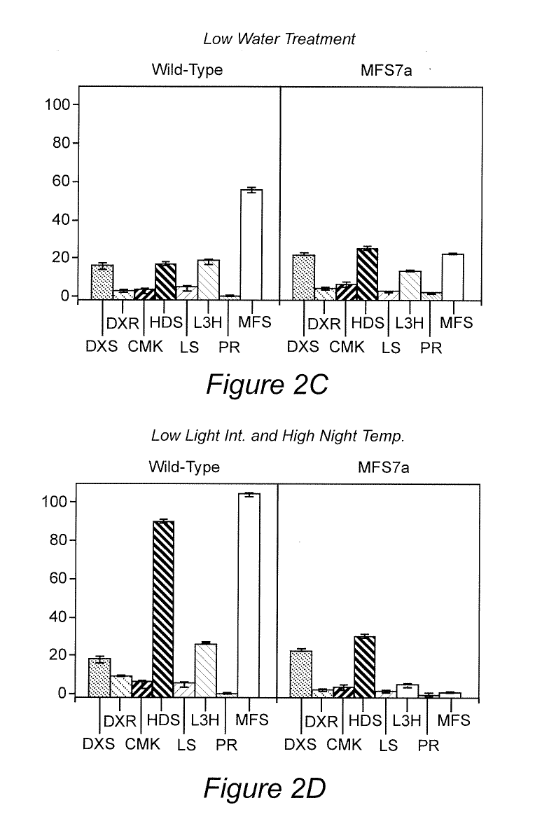

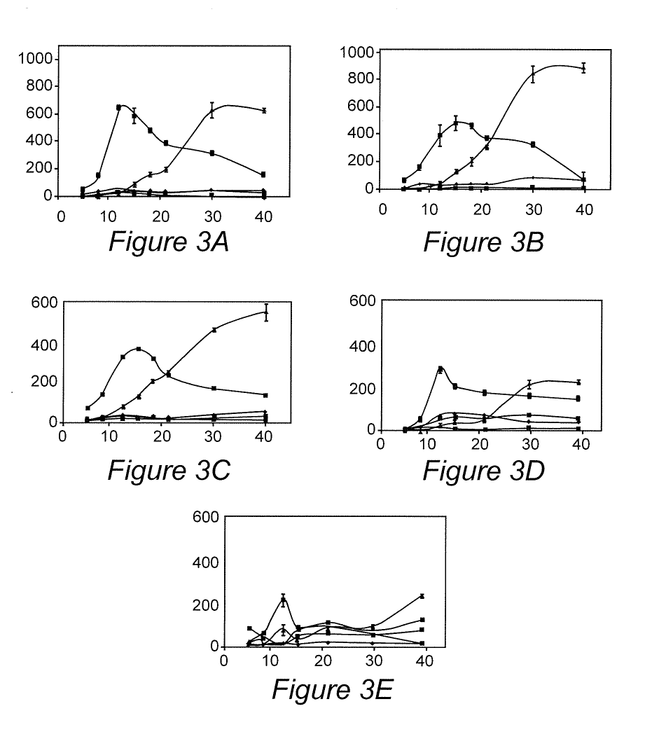

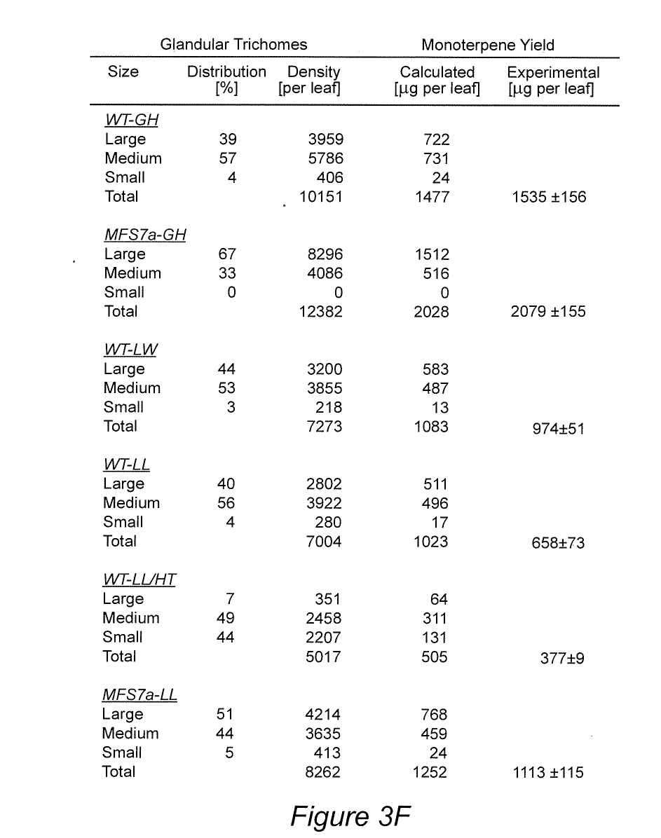

To assess gene expression patterns, secretory cells were isolated from leaves at 15 d after leaf emergence (the time of maximum essential oil biosynthetic activity), RNA was extracted (modified from Lange et al., 2000b), and the expression levels of key genes involved in determining oil quantity and composition were assayed using qPCR. In peppermint, precursors for monoterpenoid essential oils are synthesized via the plastidial mevalonate-independent pathway (Eisenreich et al, 1997) (FIG. 1A). The expression levels of the genes encoding 1-deoxy-D-xylulose 5-phosphate synthase (DXS),1-deoxy-D-xylulose 5-phosphate reductoisomerase (DXR),4-diphosphocytidyl-2C-methyl-D-erythritol kinase (CMK), and 1-hydroxy-2-methyl-2-(E)-butenyl 4-diphosphate synthase (HDS) were 4.0-fold, 1.4-fold higher, 1.4-fold and 2.4-fold higher in wild-type controls compared to MFS7 plants, respectively (FIG. 2). The increased oil yield in MFS7 plants (compared to wild-type; FIG. 3) was thus not reflected in the expression patterns of genes that code for the enzymes involved in precursor supply to the monoterpene pathway. The expression levels of relevant genes that encode enzymes of the monoterpene-specific part of the biosynthetic pathway were studied as well. The genes encoding (−)-limonene synthase (LS), (−)-limonene 3-hydroxylase (L3H) and (+)-menthofuran synthase (MFS) were expressed at high levels in wild-type controls (4.4-fold, 2.9-fold, and 7.2-fold up compared to MFS7 plants), whereas the (+)-pulegone reductase (PR) gene was expressed at very low levels (6.9-fold down compared to MFS7 plants). These expression patterns did not provide an indication as to why increased essential oil yields were detected in MFS7 plants. In contrast, the decreased amounts of (+)-pulegone and (+)-menthofuran in MFS7 plants (compared to wild-type) were indeed reflected in the expression levels of the biosynthetic genes (low MFS and high PR expression levels). Taken together, gene expression patterns appear to be consistent with monoterpene composition, but not yield, in both wild-type and MFS7 plants.

In addition to this genotype comparison, experiments were conducted with wild-type plants grown under greenhouse (control) and adverse environmental conditions. Gene expression levels of DXS, DXR, CMK, HDS, and LS followed the same pattern. In comparison to greenhouse-grown (GH) controls their expression levels decreased under drought (LW) conditions (FIG. 2). In contrast, transcript abundance of these genes increased under low light (LL) and when plants were grown at low light/high night temperatures (LL/HT) (FIG. 2). The expression levels of the L3H gene were lower in LL (2.5-fold down) and higher in LW and LL/HT samples (2.6- and 3.9-fold up, respectively) compared to GH controls (FIG. 2). The expression levels of the gene encoding PR increased slightly under all stress conditions, whereas MFS expression was notably induced under LL and LL/HT conditions (6.3- and 3.3-fold up, respectively). The measured differences in total monoterpenoid essential oil yields of wild-type plants cultivated under different environmental conditions (GH>LW>LL>LL/HT; FIG. 3) were thus not reflected in consistently lower monoterpene biosynthetic gene expression levels. However, gene expression patterns were consistent with stress-induced changes in oil composition, in particular the increased accumulation of (+)-menthofuran.

### 1.3 Maximum Glandular Trichome Size is Constant but Size Distribution and Density can Vary Substantially

Oil yield variation could potentially be caused by environment and/or genotype-dependent differences in the size of glandular trichomes, which are responsible for synthesizing and storing essential oils. To evaluate this hypothesis and enable estimations of oil yield, trichomes were divided into three different size classes: large (75-82 μm diameter), medium (65-74 μm diameter) and small (50-65 μm diameter). The volume of the essential oil-filled subcuticular cavity of mature glandular trichomes was approximated by a sphere (volume: 4/3πr³, with r=radius) minus the volume of the secretory cells (1/3πh(R²+R r+r²), with R=radius at wider end, r=radius at narrower end and h=height of frustum), multiplied by an adjustment factor (0.9) to account for the fact that the oil storage cavity also contains non-oil mucilage. Trichome oil contents were thus estimated at 2.03×10⁻⁴ μl (large-sized trichomes), 1.40×10⁻⁴ μl (average-sized trichomes) or 0.66×10⁻⁴ μl (small-sized trichomes).

At 30 d after leaf emergence the majority of glandular trichomes on WT leaves was of medium size (57%), with a substantial proportion (39%) of large-sized and a low proportion (4%) of small-sized trichomes (FIG. 3F). In contrast, the MFS7a line contained a substantially higher proportion of large-sized glandular trichomes (67% at 30 d), fewer medium-sized trichomes (33%) and no small trichomes (FIG. 3F). Leaves of greenhouse-grown plants at 30 d after emergence contained an average of 10,151 glandular trichomes, which is very similar to the estimate used thus far (10,000 trichomes per leaf). At 30 d MFS7a plants contained an average of 12,382 glandular trichomes per leaf, about 22% higher than WT. When both the size distribution and density of glandular trichomes were taken into account, the total monoterpene yield at 30 d was calculated to be 1,477 μg per leaf for WT plants (FIG. 3F), which is only 3.8% off the measured value (1,535±156 μg per leaf). Using the same approach, total monoterpene content in MFS7a plants was estimated to be 2,028 μg per leaf (2.5% off the measured value of 2,079±155 μg per leaf; FIG. 3F). These calculated monoterpene contents were about 37% higher in MFS7a than in WT, which was very close to the experimentally determined yield difference (35%).

Glandular trichome density was substantially lower on leaves of plants grown under adverse environmental conditions (7,273, 7,004 and 5,014 glandular trichomes per leaf for WT-LW, WT-LL and WT-LL/HT plants, respectively) (FIG. 3). The distribution of different sized glandular trichomes was similar in WT-GH, WT-LW, and WT-LL plants. However, at 30 d after leaf emergence, plants grown under severe stress conditions (WT-LL/HT) had a substantially higher proportion of small-size trichomes, at the expense of large-sized trichomes. Plants grown under water deficit conditions (WT-LW) produced 974±51 μg total monoterpenes per leaf, corresponding to a 60% decrease compared to greenhouse-grown controls. When peppermint plants were grown under low light intensities (WT-LL), the essential oil yield (658±73 μg monoterpenes per leaf) was roughly 2.3-fold lower than in WT-GH controls. Under severe stress conditions (WT-LL/HT), the measured essential oil yield was even lower at 377±9 μg monoterpenes per leaf.

One possible explanation for high oil yields in MFS7a plants (compared to WT) would be a higher import of carbohydrates from photosynthetic cells into non-photosynthetic glandular trichomes, thus resulting in larger precursor pools and a potentially higher oil synthesis in each trichome. This would mean that the volume of the cavity holding the essential oil would be larger. To test the hypothesis that trichomes might synthesize increased amounts of essential oil, the diameters of glandular trichomes on leaf surfaces of WT and MFS7a plants were measured. Interestingly, the maximum diameter of glandular trichomes turned out to be constant (82 μm) and independent of genotype or environmental growth conditions (data not shown). However, it was observed that the distribution of trichomes of different sizes correlated with oil yield in a genotype and environment-dependent fashion. For example, glandular trichomes on MFS7a plants emerged and matured earlier than those of wild-type plants, which was reflected in an increased proportion of large trichomes (75-82 μm diameter; 67% in MFS7a-GH vs. 39% in WT-GH; FIG. 3F). The emergence and maturation of glandular trichomes on plants grown under certain adverse environmental conditions (WT-LW and WT-LL) was similar to that in WT-GH controls and did not correlate with oil yield (FIG. 3F). Only plants grown under severe stress conditions (WT-LL/HT) had a much higher percentage of small (50-65 μm diameter) trichomes (44% in WT-LL/HT vs. 4% in WT-GH), in accordance with low oil yield (FIG. 3F). A gradient of glandular trichome sizes is generally regarded as an indicator of specific stages of leaf developmental (Turner et al., 2000). Thus, the results indicate that the architecture of peppermint glandular trichomes is a fixed parameter, whereas the program controlling trichome development is flexible. By combining the trichome distribution data with an estimate of 10,000 glandular trichomes per leaf (Colson et al., 1993), one can approximate oil yields in MFS7a-GH and WT-GH plants. Based on these calculations total monoterpene contents would be estimated to be very similar at 1,455 and 1,638 μg per leaf for WT-GH and MFS7a-GH plants, respectively (11% difference), whereas a 35% difference was detected experimentally (FIG. 3F). Using the same approach, oil yields for plants grown under adverse environmental conditions could be vastly over-estimated (estimated vs. experimental yields: WT-LW, 1,489 vs. 974±51 μg per leaf; WT-LL, 1,460 vs. 658±73 μg per leaf; WT-LL/HT: 1,007 vs. 377±9 μg per leaf), thus indicating that an additional factor needed to be considered for more accurate estimations.

While collecting glandular trichome distribution data, the number of glandular trichomes on leaves taken from WT and MFS7a plants grown under various environmental conditions was also counted. When these counts and trichome distribution data were combined, calculated oil yields for most samples deviated less than 12% from experimentally determined values (WT-GH, WT-GW, MFS7a-GH, MFS7a-LL, and L3H-GH). Larger discrepancies were observed only when plants were grown under severe stress conditions (WT-LL, estimate 36% too high and WT-LL/HT, estimate 25% too high). However, the oil yield trends (e.g., MFS7a-GH>WT-GH; MFS7a-LL>WT-LL; WT-GH>WT-LW>WT-LL>WT-LL/HT) were reflected in all approximations (FIG. 3F).

### 1.4 Glandular Trichome Density and Essential Oil Yield can be Modulated Chemically

The plant hormone methyl jasmonate (MeJA) is known to induce various defense responses in plants. Treatment of mint plants with MeJA results in significantly higher essential oil yields. These yield boosts are enabled by an increase in glandular trichome density. Thus, MeJA-mediated induction of gland cell formation may be common among plants with specialized terpenoid secretory structures, and could be utilized for increasing the yields of terpenoid essential oils and resin in numerous plants that contain specialized gland cells for terpenoid production. These results indicate that commercially relevant terpenoid essential oil yield improvements are achievable with low-dose chemical treatments.

When applied to conifer stems, MeJA causes the formation of traumatic resin ducts in certain conifers, concomitant with an induction of terpenoid resin secretion (Martin et al., 2002; Hudgins et al., 2003; Hudgins et al., 2004). Spraying Arabidopsis with MeJA leads to the induced production of trichome hairs on leaf surfaces. MeJA application also induces an increased number of glandular trichomes on tomato leaves, which emit terpenoids (Boughton et al., 2005; van Schie et al., 2007). Thus far, studies to evaluate the effects of MeJA on trichome density and essential oil yield in terpenoid accumulating plants have not been performed.

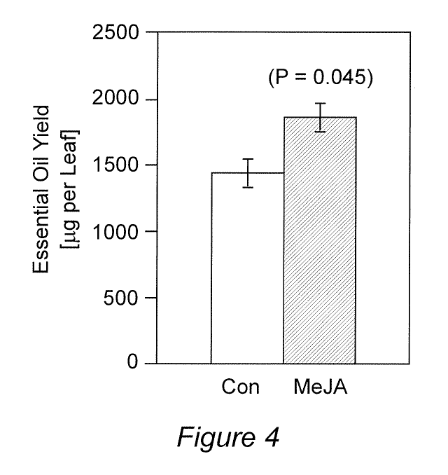

Peppermint plants were treated with low quantities of MeJA (1:4,000, v:v) dilution in water; treatment once a week with 50 ml per flat for three weeks) and monoterpene yields and glandular trichome density were measured. Leaves of MeJA-treated plants contained significantly more (24%) monoterpenoid essential oil than untreated control plants (FIG. 4), which corresponded to an increased glandular trichome density. The MeJA-mediated induction of gland cell formation might be common among plants with specialized terpenoid secretory structures, and could thus be utilized for increasing the yields of terpenoid essential oils and resin in numerous plants that contain specialized gland cells for terpenoid production. These results indicate that commercially relevant terpenoid essential oil yield improvements are achievable with low-dose chemical treatments and by modulating the expression levels of selected genes involved in terpenoid biosynthesis. It is likely that other chemicals could also be used to increase glandular trichome density and, thus essential oil yield.

### 1.7 Peppermint Shows Potential as a Versatile Platform for the Production of Oils and High-Value Small Molecules

Co-suppression of the gene encoding L3H in transgenic plants leads to the accumulation of (−)-limonene (also referred to as 1-limonene) as the principal monoterpene without detrimental effects on oil yield (Mahmoud et al., 2004). The optical isomer of 1-limonene, d-limonene, is extracted commercially from Citrus rind and is used in paint solids, to impart an orange smell to products, as a secondary cooling fluid, and, most importantly, in cleaning products. d-Limonene has excellent properties as a biodegradable solvent and can replace a wide variety of petroleum-based products, including mineral spirits, methyl ethyl ketone, acetone, toluene, glycol ethers, and fluorinated and/or chlorinated organic solvents. It has been reported that limonene can dissolve polystyrene, and there might thus be applications in recycling styrofoam as well (Noguchi et al., 1998). Since limonene (a highly reduced hydrocarbon) is combustible, it has also been considered as a biofuel additive (Freisthler, 2006). Even better combustion properties were achieved when limonene was converted to alicyclic, alkyl, and aromatic hydrocarbons (Cantrell et al., 1993), which are structurally similar to the hydrocarbon mixtures used as experimental kerosene surrogates (Dagaut et al., 2006). Thus, the enhancement of limonene production would be a desirable target for a sustainable bioenergy/biomaterials economy. Peppermint is an excellent model system for exploring the options of producing monoterpenoid hydrocarbons as precursors for biofuels and biomaterials.

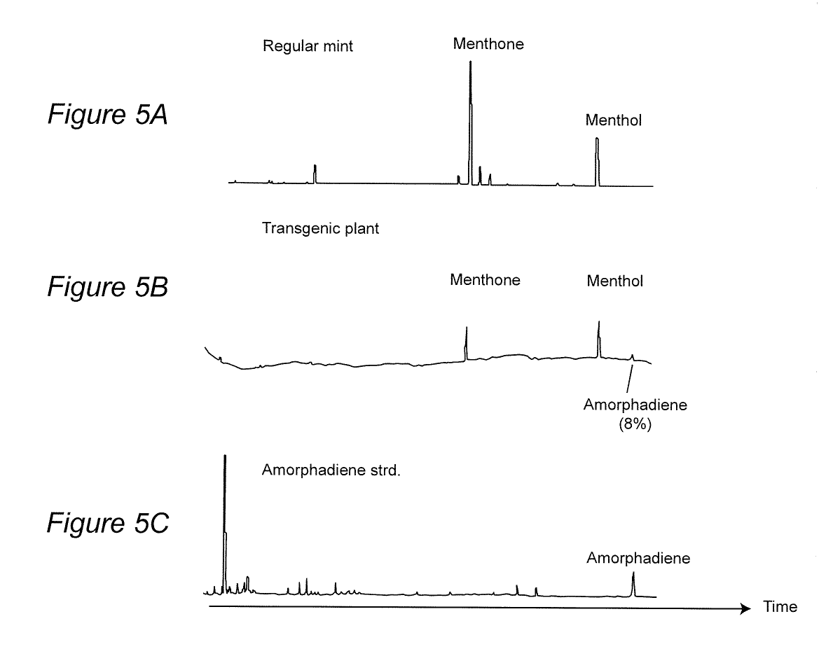

Peppermint can be utilized for the production of other valuable small molecules that are derived from the terpenoid or phenylpropanoid biosynthetic pathways, but which are not synthesized by wild-type peppermint plants. To provide examples, experiments were conducted to produce the terpenoids amorpha-1,4-diene, (−)-linalool, (+)-limonene, (−)-perillyl alcohol and/or gamma-humulene in peppermint. Using an optimized protocol, peppermint was transformed with a construct conferring ubiquitous expression of the gene encoding amorpha-1,4-diene synthase), (−)-linalool synthase, (+)-limonene synthase, (−)-limonene 7-hydroxylase or gamma-humulene synthase (amorpha-1,4-diene synthase (ADS) of Artemisia annua). The essential oil of the resulting transgenic plants contained detectable amounts of amorpha-1,4-diene, (−)-linalool, (+)-limonene, (−)-perillyl alcohol or gamma-humulene, which do not naturally accumulate in peppermint essential oil (Table 2 and FIG. 5A-C). Two different hosts were used in these transformation experiments: wild-type and a transgenic line with reduced expression levels of the gene encoding (−)-limonene 3-hydroxylase (L3H20; Mahmoud et al., 2004). Further, the volatilization of essential oil from peppermint is negligible (Gershenzon et al., 2000).

TABLE 2
Content of novel (foreign) terpenoids in transgenic peppermint plants.

| | | | | Maximum |
|---|---|---|---|---|
| | | | | Content |
| | | | | of Target |
| | | | | Compound |
| | | | Peppermint | [% of total |
| Target gene | Target Compound | Gene Source | Host Line | essential oil] |
| Monoterpene Synthases | | | | |
| (−)-Linalool Synthase | (−)-Linalool | Mentha citrata | Wild-type | 0.8 |
| (+)-Limonene Synthase | (+)-Limonene | Mentha spicata* | Wild-type | 2.9 |
| (−)-Limonene 7-Hydroxylase | (−)-Perillyl | Perilla frutescens | L3H20# | 2.1 |
| | Alcohol | | | |
| Sesquiterpene Synthases | | | | |
| Amorpha-1,4-diene Synthase | Amorpha-1,4- | Artemisia annua | Wild-type | 1.8 |
| | diene | | | |
| gamma-Humulene synthase | gamma-Humulene | Abies grandis | L3H20# | 0.7 |

*The (−)-limonene synthase gene from spearmint was subjected to site directed mutagenesis. One of the single nucleotide exchange mutants encodes a protein with (+)-limonene synthase activity.
#The host plant in these transformations was a transgenic lines with drastically reduced expression levels of (−)-limonene 3-hydroxylase (Mahmoud et al., 2004), which accumulated high levels of (−)-limonene, the substrate for (−)-limonene 7-hydroxylase.

Thus far all efforts to modulate peppermint essential oil composition and yield have relied on constructs that result in a constitutive expression of transgenes. The cell-type specific expression of transgenes in peppermint or other terpenoid/phenylpropanoid-producing plants could be achieved by using gland cell-specific promoters. Various transcription factors involved in trichome hair initiation in Arabidopsis thaliana have been characterized (Ishida et al., 2008). However, currently available evidence suggests that these regulators may induce the formation of glandular trichomes in certain plants (e.g., cotton seeds; Wang et al., 2004) but not in others (e.g., tobacco; Payne et al., 1999). In contrast, the myb transcription factor MIXTA from Antirrhinum majus, when expressed in transgenic Nicotiana tabacum plants, induced the production of trichomes on cotyledons, leaves and stems (Payne et al., 1999). Gutierrez-Alcala et al. (2005) reported that the promoter of the O-acetylserine(thiol)lyase gene from Arabidopsis thaliana conferred, when used as a promoter-GFP fusion, trichome-specific expression of the transgene in Mentha×piperita and Nicotiana tabacum. However, this promoter was not specific for glandular trichomes and is thus not suitable for utilization in the production of valuable small molecules in peppermint glandular trichomes. A trichome-specific promoter was isolated from tobacco, but this promoter conferred expression of transgenes in both glandular and non-glandular trichomes (Wang et al., 2002). Based on previous EST data sets (Lange et al., 2000b), it is known that genes involved in monoterpenoid essential oil biosynthesis are highly expressed in the secretory cells of peppermint glandular trichomes, but the activity of the encoded enzymes has not been detected in other tissues (Croteau et al., 2005). The utilization of glandular trichome-specific promoters will allow specific alteration of essential oil composition and yield without affecting metabolism in other tissues.

### 2. Exemplary Methods

### 2.1 Plant Material and Growth Conditions

Peppermint (Mentha×piperita cv. Black Mitchum) plants were grown on soil (Sunshine Mix LC1, SunGro Horticulture) in a greenhouse with supplemental lighting from sodium vapor lights (850 μmol m⁻² s⁻¹ of photosynthetically active radiation at plant canopy level) with a 16 h photoperiod and a temperature cycle of 27° C./21° C. (day/night). Transgenic plants were graciously provided by the laboratory of Dr. R. Croteau (WSU). The initial characterization of these transgenic lines was published previously: MFS7 (Mahmoud and Croteau, 2001) and L3H20 (Mahmoud et al, 2004). Plants were watered daily with a fertilizer mix (N:P:K 20:20:20, v/v/v; plus iron chelate and micronutrients). Stress experiments were performed by (1) reducing water amounts (50% of the regular volume), (2) moving plants to a growth chamber with a 16 h photoperiod at reduced light levels (300 μmol m⁻² s⁻¹ of photosynthetically active radiation at plant canopy level), and (3) combining a low light treatment (as above) with high night temperatures (30° C./30° C.; day/night).

### 2.2 Monoterpene Analysis

Leaves were directly (without prior freezing) steam-distilled and solvent-extracted using 10 mL of pentane in a condenser-cooled Likens-Nickerson apparatus (Ringer et al., 2003). Monoterpenes were identified by comparison of retention times and mass spectra to those of authentic standards in gas chromatography with mass spectrometry detection. Quantification was achieved by gas chromatography with flame ionization detection based upon calibration curves with known amounts of authentic standards and normalization to the peak area of camphor as internal standard.

### 2.3 Determination of Glandular Trichome Distribution

The distribution of glandular trichomes on peppermint leaves was evaluated using the method described by Turner et al. (2000) with minor modifications. Briefly, leaves were cut along their blade and each half was divided into three sampling zones (basal, middle and apical). Both abaxial and adaxial leaf surfaces were sampled. Transmission Electron Microscopy grids (50 mesh, 3 mm diameter; containing 12 grid squares with an enclosed area of about 0.180625 mm² each; Pelco International) were placed on leaf surfaces. Glandular trichome counting was performed in five grids per zone and on five different leaves. The total leaf area and the diameters of individual glandular trichomes were calculated based on digitized images of leaves (ImageJ; open source software developed by the National Institutes of Health) using previously described methods (Turner et al., 2000). The calculations of essential oil volume per trichome were performed as described in Rios-Estepa et al. (2008).

### 2.4 Construct Design and Agrobacterium-Mediated Transformation of Peppermint

### 2.4.1 Preparing Agrobacterium Strain

The cDNA representing the gene that encodes amorpha-1,4-diene synthase (ADS) of Artemisia annua was obtained from Dr. Peter Brodelius (Kalmar University, Sweden). A series of PCR reactions was used to generate an adapter-containing amplicon, which then recombined, using Gateway® cloning, with the pDONR201 vector (Invitrogen), thus yielding an entry clone. Cassettes containing these genes of interest were then inserted between the cauliflower mosaic virus 35 S promoter and the NOS terminator of the p*7WG2 T-DNA destination vector (Karimi et al., 2002). This vector is engineered to contain the plant selectable marker genes encoding bialaphos acetyltransferase, which confers resistance against glufosinate ammonium (Basta®). Vector plasmids were transformed into Agrobacterium tumefaciens (strains EHA105 and GV3101) by electroporation. Individual Agrobacterium colonies were picked from LB plates (1% agar containing 10 mg/l spectinomycin and 50 mg/l rifampicin) and grown at 28° C. overnight in 5 ml liquid medium (same composition as above excluding agar). A 500 μl aliquot of this culture was transferred to 50 ml fresh medium and grown to an OD₆₀₀ of 0.6-0.8 at 28° C. The suspension was centrifuged for 15 min at 3,800×g, the supernatant decanted and cell pellet suspended in 50 ml LS medium.

### 2.4.2 Transforming Peppermint Leaves

In a 250 ml Erlenmeyer flask leaves were submerged in 100 ml sterile distilled (SD) water containing 1 drop of Tween 20 and the flask was hand-shaken until the solution was visibly foamy. After adding 1 ml of a 1% (w/v) aqueous HgCl₂ solution, the flask was sealed with parafilm, shaken briefly, and leaves were incubated in the fume hood for 20 min. After decanting the incubation solution, leaves were washed with 100 ml of SD water. This rinsing was repeated three times, and acetosyringone (final concentration 0.4 mM) and the entire Agrobacterium suspension were added. While keeping leaves submerged, the upper ⅔ of the leaf blade was trimmed off and the leaf sides near the base were sliced (not cut off). The leaves were then incubated for 20 min at 25° C., removed one-by-one with sterile forceps, briefly blotted onto sterile paper towels and transferred to co-culture plates (LS medium containing 20 g/l sucrose, 2 mg/l thidiazuron (TDZ), and 4 g/l gellan gum, adjusted to pH 5.8).

### 2.5 Tissue Culture, Plant Regeneration and Analysis

After incubating leaves with Agrobacterium at 25° C. for 3-4 d in the dark, leaves were transferred to culture plates (LS medium containing 20 g/l sucrose, 4 g/l gellan gum, 4 mg/l Basta, 200 mg/l timentin, and 0.5 mg/l 6-benzyl aminopurine (BAP), adjusted to pH 5.8; designated medium M1). Plates were incubated at 25° C. for 1-2 weeks in the dark. Leaves were then transferred to culture plates containing the same medium plus 250 ml/l coconut water (designated medium M2) and incubated as above for another 2-4 weeks (transfer to new plates every 14 d). Usually callus started to form after 2-3 weeks. To induce bud formation, leaves with callus were cultivated by alternating every 1-2 weeks between M2 medium or the same medium devoid of TDZ, Basta and BAP (designated medium M3). In most cases bud formation became visible after 2-3 weeks and plates were immediately transferred to a growth chamber with light racks. Plates were covered with shade cloth to reduce irradiance to 20 μmol m⁻² s⁻¹. Two weeks after bud emergence, the leaves with callus and buds were transferred to rooting medium (LS medium containing 30 g/l sucrose, 10 mg/l naphthaleneacetic acid (NAA), 4 mg/l Basta, 4 g/l gellan gum, and 200 mg/l timentin, adjusted to pH 5.8). After an additional 2 weeks regenerating seedlings were transferred to soil and further cultivated to maturity under greenhouse conditions (25° C., 70% relative humidity, 850 μmol m⁻² s⁻¹ irradiance at canopy level). Regenerated plants were checked for the presence/absence of the transgene by PCR with genomic DNA according to routine protocols (Weigel and Glazebrook, 2002). Essential oil analyses were performed as described under 2.2.

### REFERENCES

Amelunxen F (1965) Electron microscopy analysis of glandular trichomes of Mentha piperita L (Translated from German). Planta Med 13:457-473.
Boughton A J, Hoover K, Felton G W (2005) J Chem Ecol 31:2211-2216.
Broothaerts W, Mitchell H J, Weir B, Kaines S, Smith L M, Yang W, Mayer J E, Roa-Rodríguez C, Jefferson R A (2005) Gene transfer to plants by diverse species of bacteria. Nature 433: 629-633.
Buckingham J (2000) Dictionary of Natural Products on CD-ROM, version Chapman & Hall/CRC, England CRC Press LCC.
Burbott A J, Loomis W D (1967) Effects of light and temperature on the monoterpenes of peppermint. Plant Physiol 42:20-28.
Cantrell C L, Chong N S (1993) Hydrocarbon-based fuels from biomass. U.S. Pat. No. 5,186,722.
Chaykin S., Law J., Phillips A. H., Tchen T. T. and Bloch K. (1958) Phosphorylated intermediates in the biosynthesis of squalene. Proc. Natl. Acad. Sci. USA 44: 998-1004.
Croteau R, Davis E M, Ringer K L, Wildung M R (2005) (−)-Menthol biosynthesis and molecular genetics. Naturwiss 92:562-577.
Clark R J, Menary R C (1980) Environmental effects on peppermint, I. Effect of day length, photon flux density, night temperature and day temperature on the yield and composition of peppermint oil. Aust J Plant Physiol 7:685-692.
Colby S M, Alonso W R, Katahira E J, McGarvey D J, Croteau R (1993) 4S-limonene synthase from the oil glands of spearmint (Mentha spicata). cDNA isolation, characterization, and bacterial expression of the catalytically active monoterpene cyclase. J Biol Chem 268, 23016-23024.
Crowell A L, Williams D C, Davis E M, Wildung M R, Croteau R (2002) Molecular cloning and characterization of a new linalool synthase. Arch Biochem Biophys 405, 112-121.
Dagaut P, El Bakali A, Ristori A (2006) The combustion of kerosene: experimental results and kinetic modeling using 1- to 3-component surrogate model fuels. Fuel 85, 944-956.
Diemer F, Caissard J C, Moja S, Calchat J C, Jullien F (2001) Altered monoterpene composition in transgenic mint following the introduction of 4S-limonene synthase. Plant Physiol Biochem 39:603-614.
Eisenreich W, Sagner S, Zenk M H, Bacher A (1997) Monoterpenoid essential oils are not of mevalonoid origin. Tetrahedron Lett 38:3889-3892.
Freisthler M (2006) Alternative fuel composition. U.S. Pat. No. 7,037,348.
Gelvin S B (2005) Agrobacterium-mediated plant transformation: the biology behind the “gene jockeying” tool. Microbiol. Mol Biol Rev 67: 16-37.
Gutierrez-Alcala G, Calo L, Gros F, Caissard J C, Gotor C, Romero L C (2005) A versatile promoter for the expression of proteins in glandular and non-glandular trichomes from a variety of plants. J Exp Bot 56:2487-2494.
Hudgins J W, Christiansen E, Franceschi V R (2003) Methyl jasmonate induces changes mimicking anatomical defenses in diverse members of the Pinaceae. Tree Physiol 23:361-371.
Hudgins J W, Christiansen E, Franceschi V R (2004) Induction of anatomically based defense responses in stems of diverse conifers by methyl jasmonate: a phylogenetic perspective. Tree Physiol 24:251-264.
Gershenzon J, Maffei M, Croteau R (1989) Biochemical and histochemical localization of monoterpene biosynthesis in the glandular trichomes of spearmint (Mentha spicata). Plant Physiol 89:1351-1357.
Gershenzon J, McCaskill D, Rajaonarivony J I M, Mihaliak C, Karp F, Croteau C (1992) Isolation of secretory cells from plant glandular trichomes and their use in biosynthetic studies of monoterpenes and other gland products. Anal Biochem 200:130-138.
Gershenzon J, McConkey M. E., Croteau R. B. (2000) Regulation of monoterpene accumulation in leaves of peppermint. Plant Physiol. 122, 205-214.
Ishida T, Kurata T, Okada K, Wada T (2008) A genetic regulatory network in the development of trichomes and root hairs. Annu Rev Plant Biol 59:365-386.
Krasnyansky S, May R A, Loskutov A, Ball T M, Sink K C (1999) Transformation of limonene synthase gene into peppermint (Mentha piperita L.) and preliminary studies on the essential oil profiles of single transgenic plants. Theor Appl Genet. 99:676-682.
Lange B. M., Rujan T., Martin W. and Croteau R. (2000a) Isoprenoid biosynthesis: the evolution of two ancient and distinct pathways across genomes. Proc. Natl. Acad. Sci. USA 97: 13172-13177.
Lange B M, Wildung M R, Stauber E J, Sanchez C, Pouchnik D, Croteau R (2000b) Probing essential oil biosynthesis and secretion by functional evaluation of expressed sequence tags from mint glandular trichomes. Proc Natl Acad Sci USA 97:2934-2939.
Le Novère N, Finney A, Hucka M, Bhalla U S, Campagne F, Collado-Vides J, Crampin E J, Halstead M, Klipp E, Mendes P et al. (2005) Minimum information requested in the annotation of biochemical models (MIRIAM). Nat Biotechnol 23:1509-1515.
Lucker J, Schwab W, van Hautum B, Blass J, van der PLaas L. H. W., Bouwmeester H. J., Verhoeven H. A. (2004) Increased and altered fragrance of tobacco plants after metabolic engineering using three monoterpene synthases from lemon. Plant Physiol. 134, 510-519.
Lynen F., Eggerer H., Henning U. and Kessel I. (1958) Angew. Chem. 70: 738-742.
Mahmoud S S, Croteau R B (2001) Metabolic engineering of essential oil yield and composition in mint by altering expression of deoxyxylulose phosphate reductoisomerase and menthofuran synthase. Proc Natl Acad Sci USA 98:8915-8920.
Mahmoud S S, Williams M, Croteau R (2004) Cosuppression of limonene-3-hydroxylase in peppermint promotes accumulation of limonene in the essential oil. Phytochemistry 65:547-554.
McCaskill D, Gershenzon J, Croteau R (1992) Morphology and monoterpene biosynthetic capabilities of secretory cell clusters isolated from glandular trichomes of peppermint (Mentha×piperita L.). Planta 187:445-454.
McCaskill D, Croteau R (1995) Monoterpene and sesquiterpene biosynthesis in glandular trichomes of peppermint (Mentha×piperita) rely exclusively on plastid-derived isopentenyl diphosphate. Planta 197:49-56.
Martin D, Tholl D, Gershenzon J, Bohlmann J (2002) Methyl jasmonate induces traumatic resin ducts, terpenoid resin biosynthesis, and taerpenoid accumulation in developing xylem of Norway spruce stems. Plant Physiol 129:1003-1018.
Mau C J D, Karp F, Ito M, Honda G, Croteau R (2010) A candidate cDNA clone for (−)-limonene-7-hydroxylase from Perilla frutescens. Phytochemistry 71, 373-379.
Noguchi T, Miyashita M, Inagaki Y, Watanabe H (1998) A new recycling system for expanded polystyrene using a natural solvent. Part 1. A new recycling technique. Packaging Technol. Sci. 11:19-27.
Payne T, Clement J, Arnold D, Lloyd A (1999) Heterologous myb genes distinct from GL1 enhance trichome production when overexpressed in Nicotiana tabacum. Development 126:671-682.
Ringer K L, McConkey M E, Davis E M, Rushing G W, Croteau R (2003) Monoterpene double-bond reductases of the (−)-menthol biosynthetic pathway: isolation and characterization of cDNAs encoding (−)-isopiperitenone reductase and (+)-pulegone reductase of peppermint Arch Biochem Biophys 418:80-92.
Rios-Estepa R, Turner G W, Lee J M, Croteau R B, Lange B M (2008) A systems biology approach identifies the biochemical mechanisms regulating monoterpenoid essential oil composition in peppermint. Proc Natl Acad Sci USA 105:2818-2823.
Rohloff J (1999) Monoterpene composition of essential oil from peppermint (Mentha×piperita L.) with regard to leaf position using solid-phase microextraction and gas chromatography/mass spectrometry analysis. J Agric Food Chem. 47:3782-3786.
Ruzicka L. (1953) The isoprene rule and the biogenesis of terpenic compounds. Experientia 9: 357-367.
Steele C L, Crock J, Bohlmann J, Croteau R (1998) Sesquiterpene synthases from Grand fir (Abies grandis). J Biol Chem 273, 2078-2089.
Traw M B, Bergelson J (2003) Interactive effects of jasmonic acid, salicylic acid, and gibberellin on induction of trichomes in Arabidopsis. Plant Physiol 133:1367-1375.
Turner G W, Gershenzon J, Croteau R (2000) Distribution of peltate glandular trichomes on developing leaves of peppermint (Mentha×piperita L.). Plant Physiol 124:655-664.
Van Schie C C N, Haring M A, Schuurink R C (2007) Tomato linalool synthase is induced in trichomes by jasmonic acid. Plant Mol Biol 64:251-263.
Wang E, Gan S, Wagner G J (2002) Isolation and characterization of the CYP71D16 trichome-specific promoter from Nicotiana tabacum L. J Exp Bot 53:1891-1897.
Wang S, Wang J W, Yu N, Li C H, Luo B, Gou J Y, Wang L J, Chen X Y (2004) Control of trichome development by a cotton fiber MYB gene. Plant Cell 16:2323-2334.
Wasternack C (2007) Jasmonates: an update on biosynthesis, signal transduction and action in plant stress response, growth and development. Ann Bot 1-17.

All references, patents and patent applications cited herein are hereby incorporated by reference.

We claim:

1. A genetically engineered glandular trichome-bearing plant comprising
one or more expressible genes which encode one or more proteins active in biosynthesis of at least one or more heterologous or homologous terpenes or terpenoids, wherein said heterologous or homologous terpenes or terpenoids are synthesized in glandular trichomes of said genetically engineered glandular trichome-bearing plant and stored in oil of said glandular trichomes of said genetically engineered glandular trichome-bearing plant.

2. The genetically engineered glandular trichome-bearing plant of claim 1, wherein said genetically engineered glandular trichome-bearing plant is a mint plant.

3. The genetically engineered glandular trichome-bearing plant of claim 1, wherein said one or more expressible genes is selected from the group consisting of amorpha-1,4-diene synthase, (−)-linalool synthase, (+)-limonene synthase, (−)-limonene 7-hydroxylase or gamma-humulenesynthase.

4. The genetically engineered glandular trichome-bearing plant of claim 1, wherein said one or more heterologous or homologous terpenes is selected from the group consisting of a monoterpene, a sesquiterpene, a diterpene, a triterpene, and a polyterpene.

5. The genetically engineered glandular trichome-bearing plant of claim 4, wherein said one or more heterologous or homologous terpenes is selected from the group consisting of amorpha-1,4-diene, (−)-linalool, (+)-limonene, (−)-perillyl alcohol and/or gamma-humulene.

6. The genetically engineered glandular trichome-bearing plant of claim 1, wherein said terpene or terpenoid is a homologous terpene or terpenoid.

7. The genetically engineered glandular trichome-bearing plant of claim 1, wherein said terpene or terpenoid is a heterologous terpene or terpenoid.

8. The genetically engineered glandular trichome-bearing plant of claim 1, wherein said at least one or more heterologous or homologous terpenes or terpenoids is selected from the group consisting of abietadiene, amorpha-1,4-diene, 5-epi-aristolochene, artemisinic acid, dehydroartemisinic acid, artemisinin, trans-alpha-bergamotene, beta-bisabolene, alpha- and gamma-bisabolene, (+)-bornyl diphosphate, delta-cadinene (−)-camphene, (+)-3-carene, alpha- and beta-caryophyllene, casbene, ent-cassa-12,15-diene, epi-cedrol, chrysanthemyl diphosphate, 1,8-cineole, (−)-copalyl diphosphate, ent-copalyl diphosphate, beta-cubebene, cubebol, elisabethatriene, beta-eudesmol farnesol, alpha- and beta-farnesene, geraniol, geranyllinalool, germacradienol/geosmin, germacrene A, C, and D, gossypol, alpha-gurjunene, (+)-5(6),13-halimadiene-15-ol, alpha-, beta- and gamma-humulene, epi-isozizaene, ent-kaurene, levopimaradiene (−)-limonene, (−)-isopiperitenol, (+)-limonene, (−)-linalool, longifolene, p-menthane-3,8-diol, (+)-menthofuran, (−)-menthone, (−)-menthone, cis-muuroladiene, myrcene, E-nerolidol, nootkatone, beta-ocimene, patchoulol, pentalenene, beta-phellandrene, (−)-perillyl alcohol, pimara-9(11),15-diene, syn-pimara-7,15-diene, alpha- and beta-pinene, (+)-pulegone, cis-rose oxide, ent-sandaracopimaradiene, delta-selinene, stemar-13-ene, stemodene, terpenticin, gamma-terpinene, alpha-terpineol, terpinolene, tetrahydrocannabinoic acid, trichodiene, (+)-valencene, verbenone, vetispiradiene, alpha-vetivone, viridiflorol, and alpha-zingiberene.

9. A method of producing one or more terpenes and terpenoids, comprising the steps of
selecting a glandular trichome-bearing plant;
genetically engineering said glandular trichome-bearing plant to contain and express one or more genes which encode one or more proteins active in biosynthesis of at least one or more terpenes and terpenoids, wherein said terpenes and terpenoids are synthesized in glandular trichomes of said glandular trichome-bearing plant and stored in oil of said glandular trichomes of said glandular trichome-bearing plant.

10. The method of claim 9, wherein said glandular trichome-bearing plant is a mint plant.

11. The method of claim 9, wherein said one or more genes is selected from the group consisting of amorpha-1,4-diene synthase, (−)-linalool synthase, (+)-limonene synthase, (−)-limonene 7-hydroxylase or gamma-humulenesynthase.

12. The method of claim 9, wherein said terpenes is selected from the group consisting of a monoterpene, a sesquiterpene, a diterpene, a triterpene, and a polyterpene.

13. The method of claim 12, wherein said one or more terpenes is selected from the group consisting of amorpha-1,4-diene, (−)-linalool, (+)-limonene, (−)-perillyl alcohol and/or gamma-humulene.

14. The method of claim 9, wherein said glandular trichome-bearing plant further comprises RNA sequences that inhibit expression of one or more enzymes of one or more biosynthetic pathways in said glandular trichome-bearing plant.

15. A method of producing one or more terpenes and terpenoids, comprising the steps of
growing genetically engineered glandular trichome-bearing plants, said genetically engineered glandular trichome-bearing plants comprising: one or more expressible genes which encode one or more proteins active in biosynthesis of at least one or more heterologous or homologous terpenes or terpenoids, wherein said heterologous or homologous terpenes or terpenoids are synthesized in glandular trichomes of said genetically engineered glandular trichome-bearing plants and stored in oil of said glandular trichomes of said genetically engineered glandular trichome-bearing plants; and
recovering said one or more terpenes and terpenoids from said oil of said glandular trichomes of said glandular trichome-bearing plants.

16. The method of claim 15, further comprising the step of
treating said genetically engineered glandular trichome-bearing plants with one or more jasmonates.

17. The method of claim 15, wherein said at least one or more heterologous or homologous terpenes or terpenoids is selected from the group consisting of abietadiene, amorpha-1,4-diene, 5-epi-aristolochene, artemisinic acid, dehydroartemisinic acid, artemisinin, trans-alpha-bergamotene, beta-bisabolene, alpha- and gamma-bisabolene, (+)-bornyl diphosphate, delta-cadinene (−)-camphene, (+)-3-carene, alpha- and beta-caryophyllene, casbene, ent-cassa-12,15-diene, epi-cedrol, chrysanthemyl diphosphate, 1,8-cineole, (−)-copalyl diphosphate, ent-copalyl diphosphate, beta-cubebene, cubebol, elisabethatriene, beta-eudesmol farnesol, alpha- and beta-farnesene, geraniol, geranyllinalool, germacradienol/geosmin, germacrene A, C, and D, gossypol, alpha-gurjunene, (+)-5(6),13-halimadiene-15-ol, alpha-, beta- and gamma-humulene, epi-isozizaene, ent-kaurene, levopimaradiene (−)-limonene, (−)-isopiperitenol, (+)-limonene, (−)-linalool, longifolene, p-menthane-3,8-diol, (+)-menthofuran, (−)-menthone, (−)-menthone, cis-muuroladiene, myrcene, E-nerolidol, nootkatone, beta-ocimene, patchoulol, pentalenene, beta-phellandrene, (−)-perillyl alcohol, pimara-9(11),15-diene, syn-pimara-7,15-diene, alpha- and beta-pinene, (+)-pulegone, cis-rose oxide, ent-sandaracopimaradiene, delta-selinene, stemar-13-ene, stemodene, terpenticin, gamma-terpinene, alpha-terpineol, terpinolene, tetrahydrocannabinoic acid, trichodiene, (+)-valencene, verbenone, vetispiradiene, alpha-vetivone, viridiflorol, and alpha-zingiberene.

18. A genetically engineered glandular trichome-bearing plant comprising
one or more expressible nucleic acid sequences which encode RNA that inhibits expression of one or more proteins active in biosynthesis of at least one or more terpenes or terpenoids, wherein said terpenes or terpenoids are synthesized in glandular trichomes of said genetically engineered glandular trichome-bearing plant and stored in oil of said glandular trichomes of said genetically engineered glandular trichome-bearing plant.
